(19) United States
(12) Patent Application Publication
(10) Pub. No.: US 2005/0158708 A1
(43) Pub. Date: Jul. 21, 2005

(54) Methods and compositions related to tagging of membrane surface proteins

(76) Inventors: Iris Alroy, Ness-Ziona (IL); Haim Moskowitz, Jerusalem (IL); Yuval Reiss, Kirlat-Ono (IL); Benjamin A. Shoham, Nes Ziona (IL)

Correspondence Address: FISH & NEAVE IP GROUP;ROPES & GRAY LLP, ONE INTERNATIONAL PLACE, BOSTON, MA, 02110-2624, US

(21) Appl. No.: 10/480,149

(22) PCT Filed: Jun. 6, 2002
(86) PCT No.: PCT/US02/18000

Related U.S. Application Data
(60) Provisional application No. 60/296,334, filed on Jun. 6, 2001.

(51) Int. Cl. C12Q 1/70; G01N 33/53; G01N 33/567; C07K 14/705; C07D 491/14; C07D 47/02

(52) U.S. Cl. 435/5; 435/7.2; 530/409; 546/49; 548/418; 548/525

(57) ABSTRACT

This invention relates to methods and reagents for selectively labeling membrane surface proteins using a labeling agent. The label may be used to isolate preparations of membrane surface proteins. Preparations of membrane surface proteins may be analysed by a variety of high-throughput techniques to allow rapid profiling of membrane surface protein composition.

### CROSS-REFERENCE TO RELATED APPLICATIONS

This application claims priority to U.S. Provisional Application No. 60/296,334, filed Jun. 6, 2001 and incorporated by reference herein in its entirety.

### BACKGROUND

Proteins associated with the plasma membrane constitute a significant and functionally important fraction of the proteins in a cell. Key functions, such as the communication of a cell with its environment, are largely dependent on membrane proteins. Membrane proteins are targets of choice for pharmaceuticals in part because of their exposure to the extracellular environment. Furthermore, cell surface proteins are excellent markers for use in cell sorting and identification because cells need not be damaged in order to detect these proteins.

The clinical importance of membrane proteins may be illustrated through an examination of the diagnosis and treatment of various cancers. For example, cancer therapeutics are notorious for their severe side effects, which result largely from a lack of specificity. Most cancer therapeutics target processes that are common to all growing cells and therefore cause serious damage to healthy cells in addition to cancerous cells. Substantial research has been devoted to identifying distinguishing features of cancer cells that may be used to selectively target therapeutic substances. Cancer research has also focused on the precise tailoring of therapeutic regimen to specific tumor types, with the goal of maximizing efficacy and minimizing toxicity. Improvements in cancer classification and the identification of distinctive markers for cancer types are therefore critical to advances in cancer treatment.

Cancers have traditionally been classified primarily on morphological appearance. However, tumors with similar morphology can follow significantly different clinical courses and show different responses to therapy. In a few cases, such clinical heterogeneity has been explained by dividing morphologically similar tumors into subtypes with distinct pathogeneses. Acute leukemias and non-Hodgkin's lymphomas, have been molecularly subclassified with substantial improvement in treatment efficacy. Important subclasses are likely to exist for many more tumors but have not yet to been defined by molecular markers. For example, prostate cancers of identical grade can have widely variable clinical courses. Large scale profiling of membrane proteins would provide useful “fingerprints” for the classification of cancers, and, in addition, membrane proteins unique to certain cancers could be used as targets for therapeutics or as homing signals to specifically deliver therapeutics to the appropriate cell types.

In addition to the plasma membrane, cells contain an extensive network of intracellular membranes, including the membranes surrounding the various organelles. Membrane proteins located on these intracellular are often involved in mediating interactions between the cell and the organelles, and as such represent attractive targets for research.

Membrane-embedded proteins are difficult to characterize with current methodologies. Membrane proteins are more difficult to extract due to their highly hydrophobic nature and lower solubility. The low solubility of these hydrophobic proteins, especially those of high molecular weight, gives rise to protein aggregation. Furthermore, membrane proteins are often present at relatively low abundance, making the identification of membrane proteins by, for example, microsequencing techniques, a challenging task.

It would be advantageous to have improved methods and reagents for the preparation and/or detection of cell surface protein, for example by improving the representation of cell surface proteins in protein extracts to facilitate further identification and analysis.

### SUMMARY OF THE INVENTION

In general, the invention provides methods for selectively preparing a wide range of membrane proteins, e.g. by labeling, enriching, analyzing and/or identifying membrane surface proteins, in the field of proteomics research. Preparations of membrane surface proteins generated by methods of the invention may be subjected to a variety of analytic techniques to generate profiles of these membrane surface proteins.

In one aspect, the invention provides methods for selectively labeling membrane surface proteins, and preferably cell surface proteins. In certain embodiments, methods of the invention comprise contacting a cell with a labeling agent to generate a plurality of labeled cell surface proteins. Labeling agents of the invention generally comprise a protein binding moiety and a marking moiety, wherein the protein binding moiety is capable of interacting covalently or non-covalently with a broad range of cell surface proteins, and wherein the marking moiety is useful in detecting proteins associated with the labeling agent. The protein binding moiety and marking moiety may, in certain instances, be present in a single, multifunctional moiety. Optionally, a protein binding moiety covalently binds to cysteins, glycans and/or amino groups, such as the ε-amino groups of lysine.

In certain embodiments, the properties of the labeling agent may be used to separate labeled proteins from unlabeled proteins. Labeled proteins may be processed by a variety of methods including gel electrophoresis and chromatography. Labeled proteins may also be analyzed and/or identified by techniques including, but not limited to, two-dimensional gel electrophoresis, antibody-based techniques, protein identification arrays, mass spectrometry, protein sequencing, etc. In certain embodiments, the data obtained from the identification and/or analysis of cell or membrane surface proteins forms a cell or membrane surface protein profile. Such profiles may be generated for a plurality of sample types. For example, in certain embodiments, cell and membrane surface protein profiles may be generated and compared across a variety of healthy and disordered cells, including cell lines and cultured cells. In other embodiments, profiles may also be compared for stem cells and more differentiated cells. The comparison of cell or membrane surface protein profiles will be useful for a variety of purposes including, but not limited to, diagnostics, cell identification and sorting, screening for therapeutics, identifying cell surface proteins that are indicative of certain biological conditions, etc.

In a further aspect, the invention provides methods for differential display of membrane surface proteins. Such methods generally involve selecting two or more samples to be analyzed. Each sample is treated with a labeling agent. Preferably the labeling agents are identical except that the marking moieties will be selected so as to be distinguishable. For example, a first labeling agent may comprise a first fluorescent agent modified according to the methods of the invention to become substantially membrane impermeable, and a second labeling agent may comprise a second fluorescent agent which was also made to be substantially membrane impermeable according to the method of the invention, the second fluorescent agent having fluorescent properties (e.g. excitation spectrum, emission spectrum, fluorescence efficiency, etc.) that are distinguishable from those of the first fluorescent agent. After labeling, proteins from each sample may be mixed and subjected to all further analysis together. For example, the proteins may be mixed and subjected to two-dimensional electrophoresis. In this example, the protein spots on the gel are analyzed for abundance of each fluorescent moiety to provide a direct comparison of protein abundance in the different samples. In certain embodiments differential display methods described herein may be used with more than two samples, so long as each sample is labeled with a distinguishable marker. For example, three samples may be differentially labeled with red, green and blue fluorescing moieties, mixed and analyzed to provide a differential display of the relative membrane surface protein abundance in each sample.

In a further embodiment, the invention provides reagents to be used in methods of the invention. Exemplary specific labeling agents are substantially membrane impermeable, and therefore enable selective modification of cell surface proteins. Certain labeling agents of the invention comprise a reversible bond, that facilitates removal of a substantial portion of the labeling agent from the labeled protein, which may, in certain embodiments, facilitate separation and/or identification of labeled proteins. In some embodiments of the invention the labeling agent is not a biomolecule and may therefore have a reduced tendency to form non-specific interactions with other proteins.

In certain embodiments, labeling agents of the present invention are represented by structure 1: 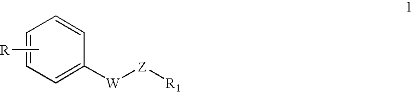
wherein:
R is present 1 to 4 times;
R is selected from the group consisting of —B(OH)₂, 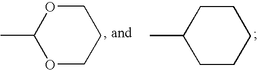
W is a linker selected from the group consisting of N(R₂)CO, CON(R₂), N(R₂)COC(R₂)₂, CON(R₂)C(R₂)₂, O, OC(R₂)₂, S, and S(R₂)₂;
Z is a spacer selected from the group consisting of a saturated or unsaturated chain up to about 6 carbon equivalents in length, unbranched saturated or unsaturated chain of from about 6 to 18 carbon equivalents in length with at least one intermediate amide or disulfide moiety, and a polyethylene glycol chain of from about 3 to 12 carbon equivalents in length;
R₁ is a reactive electrophilic or nucleophilic moiety suitable for reaction of the PDAB (phenyldiboronic acid) with a protein; and
R₂ is H, alkyl, or aryl.

In certain embodiments, the labeling agents of the present invention are of structure 1 and the accompanying definitions, wherein Z contains a disulfide moiety.

In certain embodiments, the labeling agents of the present invention are of structure 1 and the accompanying definitions, wherein R is —B(OH)₂, W is NHCO, Z is (CH₂)ₙ—S—S—(CH₂)ₙ wherein n is an integer from 1 to 6 inclusively, and R₁ is a hydrazide of structure A:

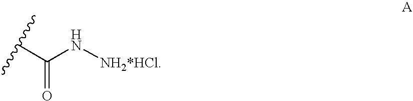

In certain embodiments, the labeling agents of the present invention are of structure 1 and the accompanying definitions, wherein R is 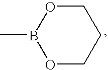
W is NHCO, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 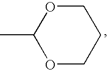
W is NHCO, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is CONH, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 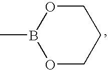
W is CONH, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 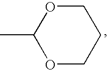
W is CONH, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is CH₂NHCO, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 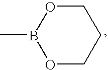
W is CH₂NHCO, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 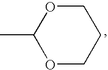
W is CH₂NHCO, Z is (CH₂)ₙ—S—S—(CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 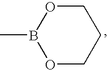
W is NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 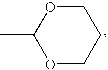
W is NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is CONH, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 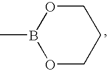
W is CONH, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 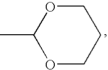
W is CONH, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is CH₂NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 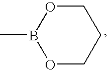
W is CH₂NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is 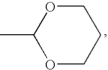
W is CH₂NHCO, Z is (CH₂)ₙ wherein n is an integer from 1 to 6 inclusively, and R₁ is a hydrazide of structure A.

In certain embodiments, the labeling agents of the present invention are of structure 1 and the accompanying definitions, wherein R is —B(OH)₂, W is CH₂NHCO, Z is (CH₂)ₙC(O)NH(CH₂)ₙ wherein n is an integer from 1 to 6 inclusively, and R₁ is a hydroxysulfo-succinimidyl ester of structure B:

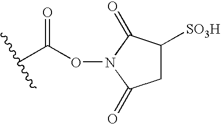

### B.

In certain embodiments, the labeling agents of the present invention are of structure 1 and the accompanying definitions, wherein R is —B(OH)₂, W is CH₂NHCO, Z is (CH₂)ₙ—S—S—(CH₂)ₙ wherein n is an integer from 1 to 6 inclusively, and R₁ is a hydroxysulfo-succinimidyl ester of structure B.

In certain embodiments, the labeling agents of the present invention are of structure 1 and the accompanying definitions, wherein R is 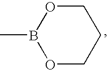
W is CH₂NHCO, Z is (CH₂)ₙC(O)NH(CH₂)ₙ wherein n is an integer from 1 to 6 inclusively, and R₁ is a hydroxysulfo-succinimidyl ester of structure B.

In certain embodiments, the labeling agents of the present invention are of structure 1 and the accompanying definitions, wherein R is 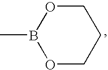
W is CH₂NHCO, Z is (CH₂)ₙ—S—S—(CH₂)ₙ wherein n is an integer from 1 to 6 inclusively, and R₁ is a hydroxysulfo-succinimidyl ester of structure B.

In certain embodiments, the labeling agents of the present invention are of structure 1 and the accompanying definitions, wherein R is 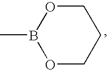
W is CONH, Z is (CH₂)₅, and R₁ is a hydroxysulfo-succinimidyl ester of structure B.

In certain embodiments, the labeling agents of the present invention are of structure 1 and the accompanying definitions, wherein R is —B(OH)₂, W is CONH, Z is (CH₂)₅, and R₁ is a hydroxysulfo-succinimidyl ester of structure B.

In certain embodiments, the labeling agents of the present invention are of structure 1 and the accompanying definitions, wherein R is 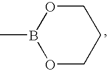
W is NHCO, Z is (CH₂)₂C(O)NH(CH₂)₅, and R₁ is a hydroxysulfo-succinimidyl ester of structure B.

In certain embodiments, the labeling agents of the present invention are of structure 1 and the accompanying definitions, wherein R is 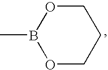
W is NHCO, Z is (CH₂)₂, and R₁ is a hydroxysulfo-succinimidyl ester of structure B.

In certain embodiments, labeling agents of the present invention are represented by structure 2: 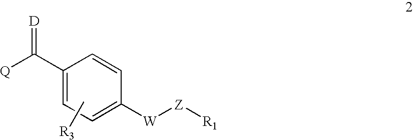
wherein:
R₃ is present 1 or 2 times and is OH;
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is a linker selected from the group consisting of N(R₂)CO, CON(R₂), N(R₂)COC(R₂)₂, CON(R₂)C(R₂)₂, O, OC(R₂)₂, S, and S(R₂)₂;
Z is a spacer selected from the group consisting of a saturated or unsaturated chain up to about 6 carbon equivalents in length, unbranched saturated or unsaturated chain of from about 6 to 18 carbon equivalents in length with at least one intermediate amide or disulfide moiety, and a polyethylene glycol chain of from about 3 to 12 carbon equivalents in length;
R₁ is a reactive electrophilic or nucleophilic moiety suitable for reaction of the PDAB (phenyldiboronic acid) with a protein; and
R₂ is H, alkyl, or aryl.

In certain embodiments, the labeling agents of the present invention are of structure 2 and the accompanying definitions, wherein Z contains a disulfide moiety.

In certain embodiments, the labeling agents of the present invention are of structure 2 and the accompanying definitions, wherein R is present one time W is NHCO, Z is (CH₂)ₙ—S—S—(CH₂)ₙ wherein n is an integer from 1 to 6 inclusively, Q is OR₂ and R₁ is a hydrazide of structure A:

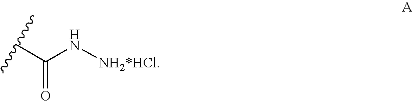

In certain embodiments, the labeling agents of the present invention are of structure 2 and the accompanying definitions, wherein R is present one time, W is NHCO, Z is (CH₂)ₙ—S—S—(CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present two times, W is NHCO, Z is (CH₂)ₙ—S—S—(CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present two times, W is NHCO, Z is (CH₂)ₙ—S—S—(CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present one time, W is CONH, Z is (CH₂)ₙ—S—S—(CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present one time, W is CONH, Z is (CH₂)ₙ—S—S—(CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present two times, W is CONH, Z is (CH₂)ₙ—S—S—(CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present two times, W is CONH, Z is (CH₂)ₙ—S—S—(CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present one time, W is CONH, Z is (CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present one time, W is CONH, Z is (CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present two times, W is CONH, Z is (CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present two times, W is CONH, Z is (CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present one time, W is NHCO, Z is (CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present one time, W is NHCO, Z is (CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present two times, W is NHCO, Z is (CH₂)ₙ wherein n is an integer from 1 to 6 inclusively, Q is OR₂, and R₁ is a hydrazide of structure A.

In certain embodiments, the labeling agents of the present invention are of structure 2 and the accompanying definitions, wherein R is present two times, W is NHCO, Z is (CH₂)ₙ wherein n is an integer from 1 to 6 inclusively, Q is NHOR₂, and R₁ is a hydrazide of structure A.

In certain embodiments, the labeling agents of the present invention are of structure 2 and the accompanying definitions, wherein R is present one time W is NHCO, Z is (CH₂)ₙ—S—S—(CH₂)ₙ wherein n is an integer from 1 to 6 inclusively, Q is OR₂ and R₁ is a hydrazide of structure B:

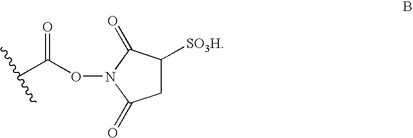

In certain embodiments, the labeling agents of the present invention are of structure 2 and the accompanying definitions, wherein R is present one time, W is NHCO, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present two times, W is NHCO, Z is (CH₂)ₙ—S—S—(CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present two times, W is NHCO, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present one time, W is CONH, Z is (CH₂)ₙ—S—S—(CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present one time, W is CONH, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present two times, W is CONH, Z is (CH₂)ₙ—S—S—(CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present two times, W is CONH, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present one time, W is CONH, Z is (CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present one time, W is CONH, Z is (CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present two times, W is CONH, Z is (CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present two times, W is CONH, Z is (CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present one time, W is NHCO, Z is (CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present one time, W is NHCO, Z is (CH₂)ₙ wherein n is an integer from 1 to 6 inclusively, Q is NHOR₂, and R₁ is a hydroxysulfo-succinimidyl ester of structure B.

In certain embodiments, the labeling agents of the present invention are of structure 2 and the accompanying definitions, wherein R is present two times, W is NHCO, Z is (CH₂)ₙ wherein n is an integer from 1 to 6 inclusively, Q is OR₂, and R₁ is a hydroxysulfo-succinimidyl ester of structure B.

In certain embodiments, the labeling agents of the present invention are of structure 2 and the accompanying definitions, wherein R is present two times, W is NHCO, Z is (CH₂)ₙ wherein n is an integer from 1 to 6 inclusively, Q is NHOR₂, and R₁ is a hydroxysulfo-succinimidyl ester of structure B.

Various embodiments are described in the claims, and all such embodiments hereby incorporated into the specifcation.

The practice of the present invention will employ, unless otherwise indicated, conventional techniques of chemistry, cell biology, cell culture, molecular biology, transgenic biology, microbiology, recombinant DNA, and immunology, which are within the skill of the art. Such techniques are explained fully in the literature. See, for example, “Bioconjugate Techniques”, G T Hermanson, Academic Press (1996); Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1: A flowchart illustrating exemplary methodologies for the profiling of cell surface proteins.

FIG. 2: A flowchart illustrating exemplary methodologies for the multiple labeling and profiling of membrane surface proteins.

FIG. 3: Exemplary labeling agents comprising a phenylboronic acid (“PBA”) type marking moiety.

FIG. 4: Exemplary labeling agents comprising a PBA type marking moiety.

FIG. 5: Exemplary labeling agents comprising a PBA type marking moiety.

FIG. 6: Exemplary labeling agents comprising a salicylhydroxamic acid (“SHA”) marking moiety.

FIG. 7: Exemplary labeling agents comprising an SHA marking moiety.

FIG. 8: Exemplary labeling agents comprising an SHA marling moiety.

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “biological state” is used herein to refer to essentially any biologically relevant characteristic of a cell or tissue sample. “Biological state” may refer to the presence or absence of a disease condition, a tissue type, a developmental stage, an effect on a tissue or cell caused by a therapeutic or other biologically active compound, etc.

A “cell sample” is any sample obtained from a biological source and containing cells. Cell samples are intended to encompass, without limitation, solid or semi-solid tissue samples (eg. tumor biopsy, skin scraping, stool sample, etc.) as well as fluid samples (eg. blood, urine, cerebro-spinal fluid, saliva etc.). Cell samples also include cultured cells and cell lines. A “test cell sample” is a cell sample for which it is desirable to characterize a biological state. A “reference cell sample” is a cell sample which has been characterized with respect to a biological state. A “diseased cell sample” is a cell sample affected by a disorder, disease or abnormal state, including genetically or otherwise altered cell lines or cultured cells.

A “cell surface protein” is used herein to mean any protein that is exposed to the extracellular environment and associated with the membrane. Cell surface proteins include, but are not limited to, integral membrane proteins (i.e. proteins with one or more transmembrane domains), membrane-anchored proteins (i.e. proteins attached to the membrane through a lipophilic anchor), and membrane-associated proteins (i.e. proteins that have some affinity for the membrane but are not covalently attached to a moiety that is inserted in the membrane).

A “cell surface protein profile” or “membrane surface protein profile” is used herein to indicate an aggregate of information regarding a preparation of cell or membrane surface proteins. A profile will comprise, at minimum, information regarding the presence or absence of such proteins. More typically, a profile will comprise information regarding the presence or absence of a plurality of such proteins. In addition, a profile may contain other information about each identified protein, such as relative or absolute amount of protein present, the degree of post-translational modification, membrane topology, three-dimensional structure, isoelectric point, molecular weight, etc. A “test cell surface protein profile” is a cell surface protein profile obtained from a test cell sample. A “reference cell surface protein profile” is a cell surface protein profile obtained from a reference cell sample.

A “chimeric protein” or “fusion protein” is a fusion of a first amino acid sequence encoding a polypeptide with a second amino acid sequence heterologous to the first amino acid sequence.

“Closed membrane structures” are membrane structures that are topologically configured so as to create at least two chemically distinguishable compartments: an inside and an outside. Closed membrane structures include, but are not limited to, membrane vesicles (whether artificial or obtained from a biological sample), cells and organelles such as mitochondria, lysosomes, peroxisomes, chloroplasts, endosomes, etc.

The term “comprising” is used in the inclusive, open sense, meaning that additional elements may be included.

The term “divalent ion chelator” is used herein to refer to compounds that bind with high affinity (having a dissociation constant under normal biochemical conditions of less than about 10-10 nM) to one or more divalent ions, such as, for example, Ca2+, Mg2+, Fe2+, etc.

The term “including” is used herein to mean “including but not limited to”. “Including” and “including but not limited to” are used interchangeably.

The term “isolated”, as used herein with reference to the subject proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof.

A “marking moiety” is essentially any molecular moiety that can be used, directly or indirectly, to detect those proteins that are bound to a labeling agent, e.g. by providing a directly detectable moiety such as a fluorescent moiety, a radioactive moiety, etc., or by serving as an affinity capturing agent, such as a biotin (for capture by, e.g., an avidin), a sulfhydryl (for capture by e.g., another sulfhydryl), a phenylboronic acid (“PBA”) (for capture by, e.g., a salicylhydroxamic acid), a salicylhydroxamic acid (“SHA”) (for capture by, e.g., a phenylboronic acid), etc. Marking moieties are joined to protein binding moieties to form labeling agents.

A “membrane surface protein” is used herein to refer to a protein that is exposed to the environment on the external side of a closed membrane structure. Membrane surface proteins include, but are not limited to, integral membrane proteins (i.e. proteins with one or more transmembrane domains), membrane-anchored proteins (i.e. proteins attached to the membrane through a lipophilic anchor), and membrane-associated proteins (i.e. proteins that have some affinity for the membrane but are not covalently attached to a moiety that is inserted in the membrane).

The terms “proteins” and “polypeptides” are used interchangeably herein.

A “protein binding moiety” or “binding moiety” is a molecular moiety that is capable of interacting, covalently or non-covalently, with a broad range of proteins. Exemplary classes of protein binding moieties include lectins, and amide- or thiol-reactive agents. Protein binding moieties are joined with marking moieties to form labeling agents The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

The term “reversible bond” includes covalent bonds that are reversible under conditions that are relatively gentle with respect to polypeptides (e.g. a pH that does not cause peptide bond hydrolysis, reducing conditions that do not cause substantial modifications to amino acid side chains other than a sulfhydryl, etc.). A disulfide bond is an exemplary reversible bond.

The term “selective” as used in reference to the tagging of membrane surface proteins, is intended to indicate that the labeling agent, when used according to methods described herein, primarily labels membrane surface proteins and not other types of proteins, such as cytoplasmic proteins. “Selective” may indicate that more than 70% of tagged proteins are membrane surface proteins (i.e. the mass of tagged proteins that are known to be membrane surface proteins divided by the mass of tagged proteins is greater than 0.7). In other embodiments, “selective” indicates that more than 80%, more than 90% or more than 95% percent of tagged proteins are membrane surface proteins. The percentage of tagged proteins that are membrane proteins may be assessed by examining a representative sample of the tagged proteins.

The term “separating” is used herein to refer to any of a variety of methods that may be used to resolve a complex mixture of proteins into simpler mixtures, or pure proteins, for identification. Separation may include, but is not limited to, chromatography, gel electrophoresis (for example two-dimensional gel electrophoresis), adherence to a protein identification array, and/or differential precipitation (or other methods of protein purification), etc. For example, resolution of a mixture of proteins into spots (some will be distinct, others will be less so) by two-dimensional gel electrophoresis is considered “separating”. As a further example, placing a mixture of proteins on a protein identification array comprising an ordered array of antibodies is considered “separating”, because different proteins adhere to different positions on the array.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

The term “substantially membrane impermeable” as used in reference to labeling agents means that the labeling agent, when employed in methods disclosed herein, is effective for selectively tagging membrane surface proteins.

The term “test compound” as used herein is meant to include, but is not limited to, peptides, nucleic acids, carbohydrates, small organic molecules, natural product extract libraries, and any other molecules (including, but not limited to, chemicals, metals and organometallic compounds).

3. Membrane Labeling Methods

In certain aspects, the invention provides reagents and methods for selectively tagging proteins that are exposed to the extracellular environment. In certain embodiments, selective tagging may be accomplished through the use of a labeling agent. In general, labeling agents have the following properties: (1) the ability to interact relatively non-specifically, and covalently or non-covalently, with a wide range of proteins; and (2) an inability to penetrate the cell membrane or an inability to stably interact with intracellular proteins (i.e. a labeling agent that penetrates the cell but is destroyed or rendered inoperative by the intracellular environment may be effective for selectively labeling cell surface proteins). For example, lectins bind to glycoproteins and show some discrimination between glycoproteins. Labeling agents of the invention generally comprise a protein binding moiety and a marking moiety, wherein the protein binding moiety is capable of interacting covalently or non-covalently with a broad range of cell surface proteins, and wherein the marking moiety is useful in identifying proteins associated with the labeling agent. The protein binding moiety and marking moiety may, in certain instances, be present in a single, multifunctional moiety.

In certain embodiments, the protein binding moiety forms one or more covalent bonds with proteins, often by reacting with, for example, α- or ε-amine, thiols and glycans. Examples of such protein binding moieties are known in the art and, in view of this specification, one of skill in the art would be able to select an appropriate moiety for incorporation into a labeling agent. In general there are three major classes of moieties that form covalent bonds with amines: succinimidyl esters (eg. N-hydroxysuccinimide, or NHS) and including sulfosuccinimidyl esters, isothiocyanates, and sulfonyl chlorides. Other amine-reactive moieties include, but are not limited to, dichlorotriazines, aryl halides and acyl azides. Thiol reactive moieties include, but are not limited to, haloalkyls (eg. iodoacetamides), maleimides, and bimanes (eg. monobromotrimethylammoniobimane, p-sulfobenzoyloxybromobimane). In general, thiol-reactive moieties show preference for interaction with cysteine residues, with lesser interaction with methionines. Maleimides have higher selectivity for cysteine over methionine than do the haloalkyls.

In further embodiments, the protein binding moiety binds non-covalently to a broad range of proteins. For example, lectins are a class of proteins that bind to glycoproteins through the interaction with one or more sugar subunits. Because glycoproteins share many of the same oligosaccharide modifications, lectins tend to bind to a broad array of proteins and are thus suitable as relatively non-specific labeling agents. Exemplary lectins include, but are not limited to, concanavalin A, phytohemagglutinin, isolectin GS-IB4 from Griffonia simplicifolia, lectin HPA from Helix pomatia, lectin SBA from Glycine max, lectin PNA from Arachis hypogaea, lectin GS-II from Griffonia simplicifolia, etc.

In certain embodiments, marking moieties are members of a specific binding pair, meaning that the marking moiety interacts specifically with a binding partner. As an illustrative example, biotin and streptavidin form a specific binding pair. It is preferable that the specific binding pair interact with a dissociation constant (K_{D}) of less than about 10⁻⁶, and, more preferably, less than about 10⁻⁹. Other exemplary specific binding pairs include, but are not limited to, metals (including partially liganded metals) and metal binding agents (eg. nickel and polyhistidine, divalent cations and EDTA, iron and hemoglobin, etc.), chitin and chitin binding protein, cellulose and cellulose binding protein, glutathione and glutathione-S-transferase, an antibody-antigen pair, a magnetic metal and a magnet, etc. Another exemplary specific binding pair is PHB (or modifications thereof that retain the ability to interact with SHA) and SHA (or modifications thereof that retain the ability to interact with PHB), which form a covalent bond under relatively mild conditions, the resultant covalent complex being stable even when exposed to strong chaotropic or protein denaturing agent.

In further embodiments, marking reagents provide a novel functional group that may be reacted with additional labeling agents at a later time. In certain exemplary embodiments, a marking reagent provides a thiol group that can react with a second labeling agent that is thiol reactive. Accordingly, in one aspect, membrane surface proteins may be contacted with a first labeling agent that comprises an amine-reactive protein binding moiety and a marking reagent that has a disulfide bond. The labeling reagent attaches to exposed amines. Subsequently, the disulfide bond may be reduced, to yield an exposed thiol. The proteins may then contacted with a second labeling agent that has a desired marking moiety and a thiol-reactive protein binding moiety. In a sense, the method permits the conversion of amines into thiols so that a labeling agent containing a thiol-reactive moiety can be used to label proteins at positions normally having amines. This procedure is advantageous in part because it greatly increases the utility of labeling agents having thiol-reactive moieties. Proteins generally have far more free amines than free thiols, and accordingly thiol-reactive labeling agents tend to label fewer proteins and have a weaker signal per protein. By providing thiol groups at positions that normally have amines, it is possible to achieve stronger and more general labeling with thiol reactive groups.

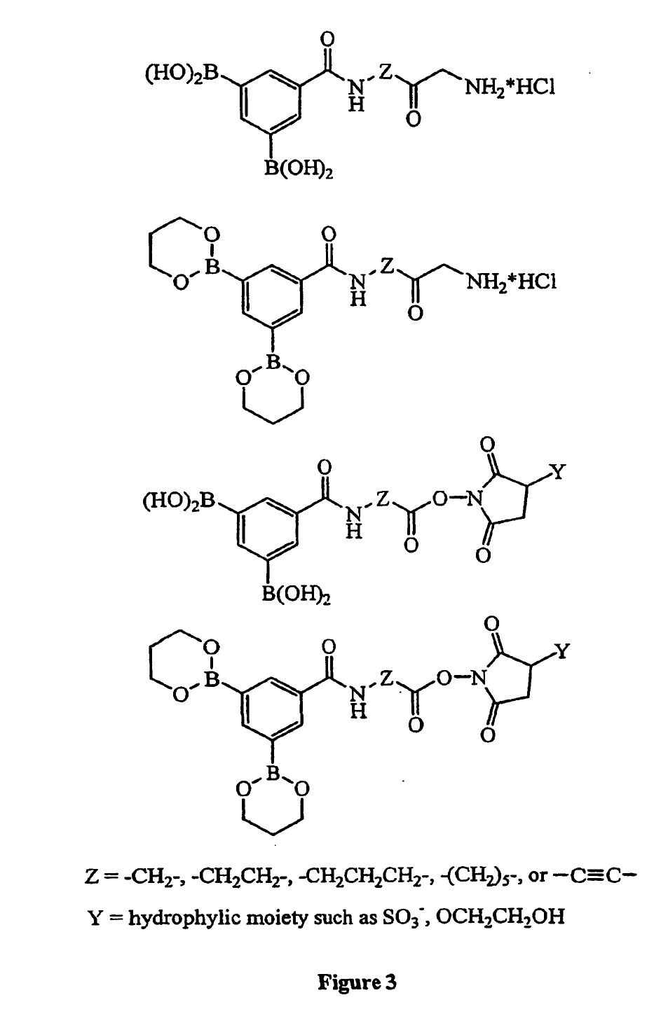

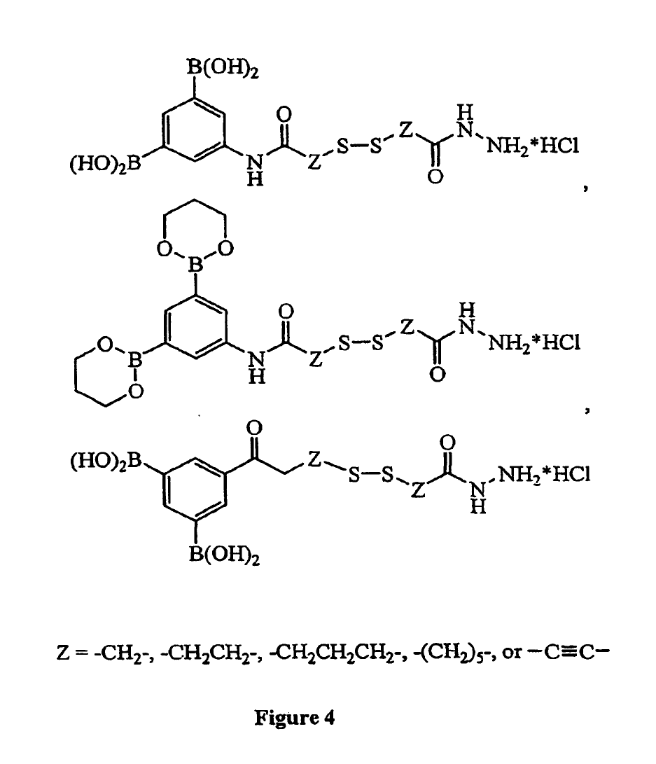

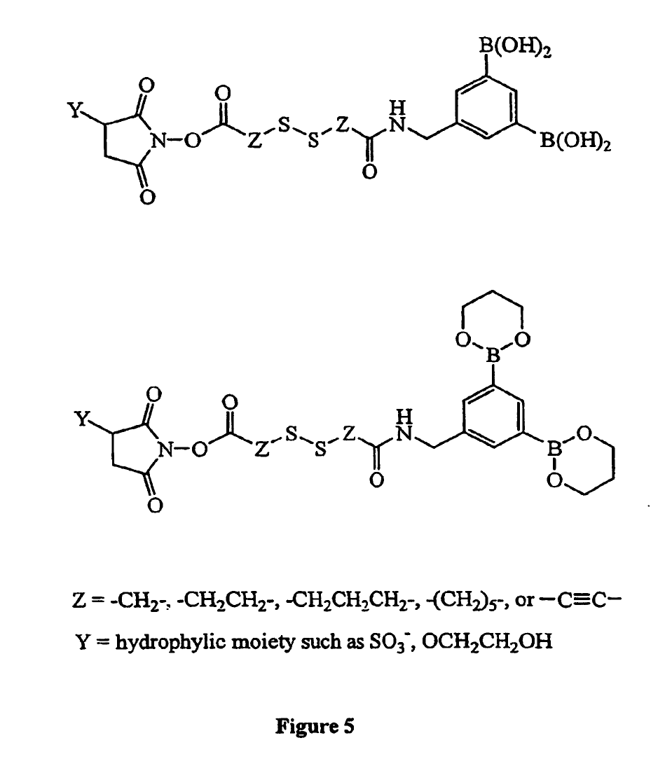

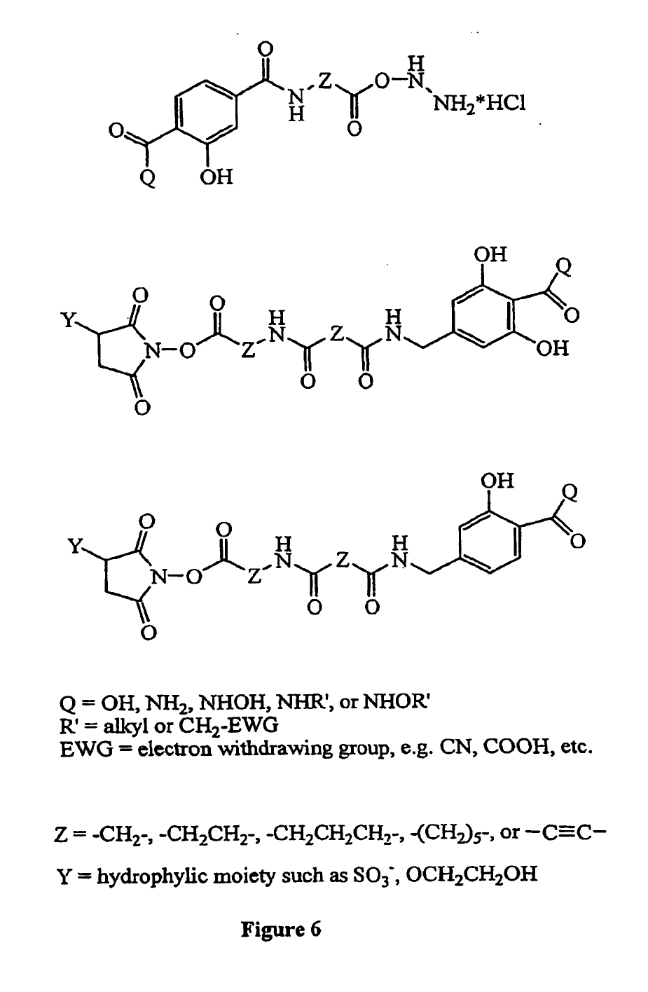

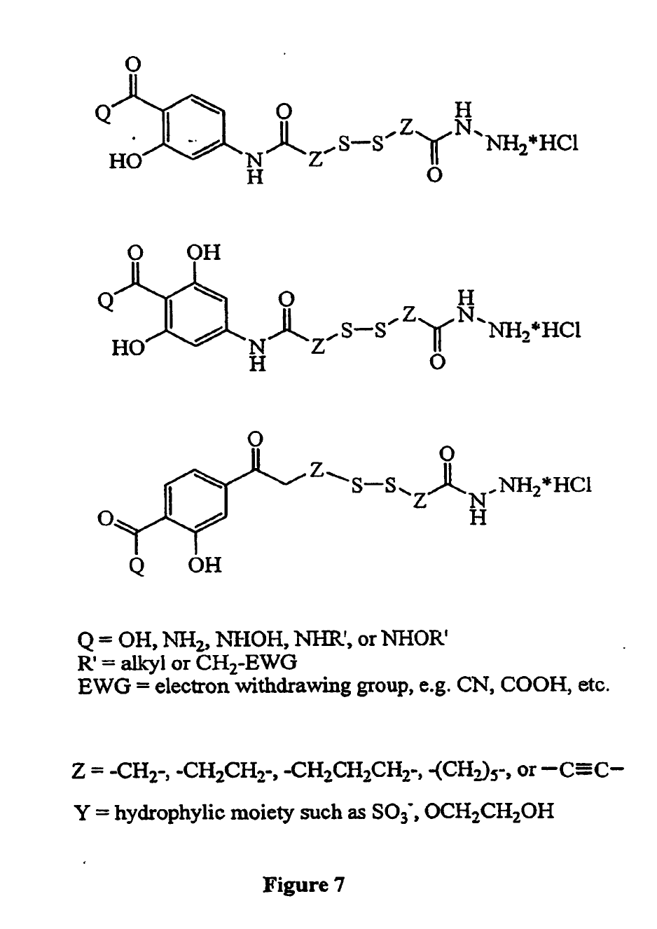

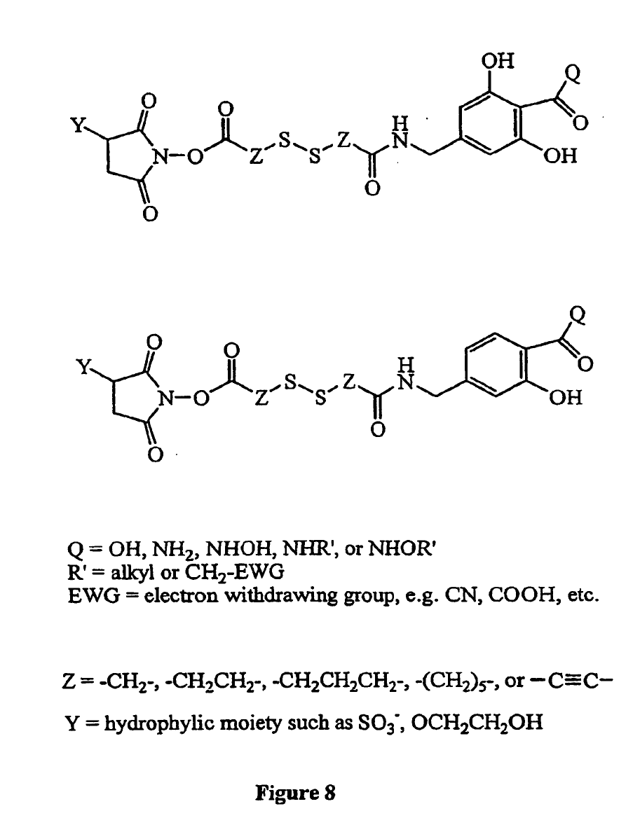

In certain embodiments, a labeling agent comprises a marking moiety that comprises a phenylboronic acid (or modifications thereof that retain the ability to interact with SHA) or a salicylhydroxamic acid (or modifications thereof that retain the ability to interact with PHB). A marking moiety comprising a PHB may be captured by an agent comprising an SHA. The agent comprising the SHA may include essentially any useful additional element, such as a fluorescent label, a member of a specific binding pair, etc. Likewise, a marking moiety comprising an SHA may be captured by an agent comprising a PHB. The agent comprising the PHB may include essentially any useful additional element, such as a fluorescent label, a member of a specific binding pair, etc. PBA and SHA react to form a strong complex in moderate conditions and in a biologically-compatible buffer environment. The link formed between a PHB and an SHA is resistant to dissolution, and proteins labeled with such a complex may be subjected to treatment with chaotropic agents that are useful, for example, for membrane solubilization. Such chaotropic agents are harmful to many labeling systems, such as a biotin/avidin system. Labeling agents of this type may include, as a binding moiety, a reactive group that is, for example, reactive with a sugar group, an amine and/or a sulfhydryl. Exemplary binding moieties include N-hydroxy-Succinamide (“NHS”) or hydrazide. The hydrazide moiety is useful, for example, for relatively non-specific tagging of glycoproteins. The amount of tagging will depend on the amount of oxidation on the glycan and may be controlled by gradual oxidation of the glycans. Gradual tagging may be used, for example, to tag proteins with two types of labeling agents, such as a first labeling agent that is useful for direct detection of the labeled proteins and a second labeling agent that is useful for affinity capture of the labeled proteins. The amount of oxidation on a glycoprotein may be controlled by, for example, manipulating the concentration of an oxidant such as NaIO₄, manipulating the time of exposure to an oxidant and the temperature. Alternatively, the tagging can be performed after enzymatic oxidation. Exemplary labeling agents of the PBA or SHA types include those presented in FIGS. 3-8. Certain exemplary labeling agents of these types are available from Prolinx, Inc. (Bothell, Wash.). Exemplary labeling agents of these types, and methods for preparation are described in U.S. Pat. No. 5,777,148.

In certain embodiments, the invention provides novel labeling agents based on the PBA and SHA structures described herein, as well as additional reagents that are specifically suitable for performing certain methods of the invention. In one embodiment, the invention provides PBA or SHA-based labeling agents comprising a disulfide bond positioned within an aliphatic chain. The disulfide bond enables removal a substantial portion of the reagent from the tagged protein under gentle reducing conditions. The amount of labeling agent removed depends on the position of the disulfide bond within the aliphatic chain. For example, the disulfide bond may be positioned to leave a tag of approximately 89 Da or smaller. Furthermore, the disulfide group decreases the membrane permeability of the labeling agent and facilitates many further manipulations such as detection with mass spectroscopy, resolution by gel electrophoresis, etc. In a further exemplary class of novel molecules, a disulfide bond is incorporated into the aliphatic carbon chain of PBA- or SHA-based molecule comprising hydrazide as its binding moiety. This agent, as noted above, is useful, for example, for relatively non-specific tagging of glycoproteins and may be used in gradual labeling and multiple labeling protocols. Labeling agents of this type are advantageous, in part, because the disulfide bond may be reversed to leave only a minimal group on the labeled proteins. The ability to remove a substantial portion of the labeling agent may, in certain embodiments, facilitate protein separation and/or identification. This labeling agent is not a biomolecule and therefore it has a reduced tendency to interact non-specifically with other proteins. In a further aspect, the invention provides PBA- and SHA-based labeling agents comprising one or more additional hydrophilic moieties. In general, the inventive labeling agents comprise sufficient hydrophilic moieties to be substantially membrane impermeable. Exemplary hydophilic moieties include polyethyleneglycols and charged groups such as sulfonates. In certain exemplary embodiments, a hydrophilic moiety is bonded to the NHS active ester portion of a labeling agent. Substantially membrane impermeable labeling agents comprising a PBA or SHA type group may, depending on the embodiment, have a number of previously unappreciated advantages. For example, in some aspects the use of the method of the invention reduces non-specific interactions caused by endogenous biological molecules such as biotin. Since this technology is chemically based it is free of the limitation of denaturation of the avidin/biotin complex and therefore it is possible to work with strong chaotropic agents and other denaturating solubilization techniques. This enables a specific and improved tagging of membrane proteins. In certain aspect, the use of these labeling agents and methods of the invention also enable solubilizing tagged membrane proteins with strong buffers such as urea, thiourea and detergents.

The interaction between the marking moiety and a specific binding partner can be used in a variety of ways to identify those proteins that are labeled with the labeling agent. For example, labeled proteins may be separated from unlabeled proteins by affinity purification using the specific binding partner. The specific binding partner would typically be affixed to a solid, semi-solid or insoluble substrate (most commonly a polymeric substance formed into small beads) and exposed to the mixture of labeled and unlabeled proteins. Labeled proteins will tightly associate with the substrate through the interaction between the affixed binding partner and the marking moiety of the labeling agent. In view of this specification, many variations on the general methods of separation using the binding partner are known to those of skill in the art.

In another example, the specific binding partner may be modified with a detectable reagent (eg. fluorescent, radioactive, colored) and then exposed to a mixture of labeled and unlabeled proteins. Those proteins that are bound to a labeling agent will bind to the detectable binding partner and can then be detected. Labeled and unlabeled proteins may also be separated (for example by gel electrophoresis or chromatography) and then detected using the specific binding partner.

In a further embodiment, labeled membrane surface proteins may be affixed to a solid surface to form an array of the labeled proteins. For this embodiment, the solid surface is prepared by affixing an agent that binds to a marking moiety to be introduced onto the labeled membrane surface proteins. The membrane surface proteins are selectively labeled and contacted with the prepared surface, thereby becoming bound to the solid surface to make an array of labeled membrane surface proteins. For example, if the labeling agent comprises an SHA-type marking moiety, then the solid surface is prepared with a PBA-type moiety. As another example, if the labeling agent comprises a disulfide bond to be reduced so as to reveal a free sulfhydryl, then the solid surface may be prepared with a sulfhydryl reactive reagent. A solid surface may be a MALDI-TOF MS target (the solid support of the samples to be tested in the instrument). Such MALDI targets can be the Ciphergen (Freemont, Calif.) instrument or other MALDI-TOF instruments. After attachment to the solid surface, the proteins may be washed with a buffer, such as ammonum bicarbonate 25 mM pH=8.5, to reduce non-specific binding and to equilibrate the pH to between 7 and 9, and optionally approximately 8.5. If desired, proteins in the array may be analyzed by mass spectrometry. For example, the proteins may be digested with a protease such as trypsin. The next step is adding MALDI matrix like alpha-cyano (or equivalent) and analyzing the mixture of peptides using the MALDI-TOF or MALDI-TOF/TOF instruments.

In yet another embodiment, the marking moiety is fluorescent. In certain embodiments, a fluorescent marking moiety is substantially membrane impermeable, and optionally the membrane permeability is decreased by modifying the fluorescent moiety with one or more hydrophilic elements, such as polyethylene glycols and/or charged groups such as sulfonates. Exemplary fluorescent moieties, presented here with no intent to be comprehensive or limiting, include fluoresceins, benzoxadioazoles, coumarins, eosins, Lucifer Yellow, pyridyloxazoles, flavins, peridinin-chlorophyll a, phycoerythrins, phycocyanins, and rhodamines. These and many other exemplary fluorescent moieties may be found in the Handbook of Fluorescent Probes and Research Chemicals (2000, Molecular Probes, Inc.). Exemplary fluorescent coumarins are shown below. In certain embodiments, a method of the invention employs a labeling reagent comprising an SHA-type group as a marking moiety, reacting the labeling agent with a closed membrane structure and then reacting the labeled proteins (having the SHA-type group attached) with a PBA-type group that is attached to a fluorescent moiety, such as a fluorescent coumarin. In additional embodiments, the PBA-type group may be part of the labeling agent and the SHA attached to a fluorescent moiety. As will be appreciated by one of skill in the art, the fluorescent coumarins are presented coupled to an exemplary amine-reactive protein binding moiety. It is understood that any of a variety of protein binding moieties may be substituted. In preferred embodiments, the protein binding moiety is a succinimidyl ester that has been modified to increase the hydrophilicity of the labeling agent, optionally by adding a sulfonate. The fluorescent coumarins below are numbered according to the optimal excitation wavelength and are commercially available from Molecular Probes, Inc. under the name Alexa Fluor®.

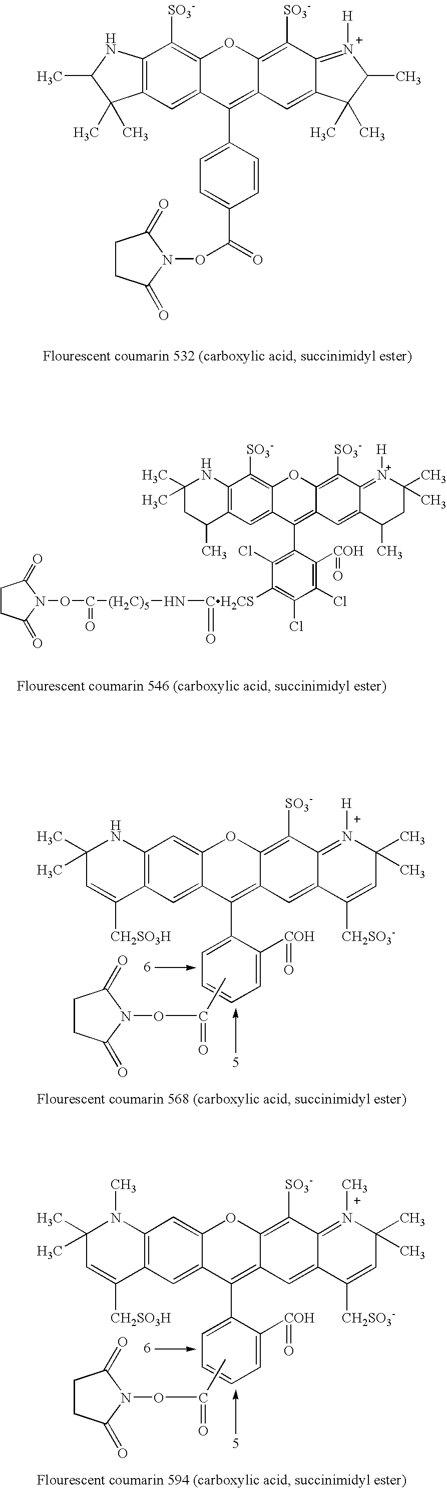

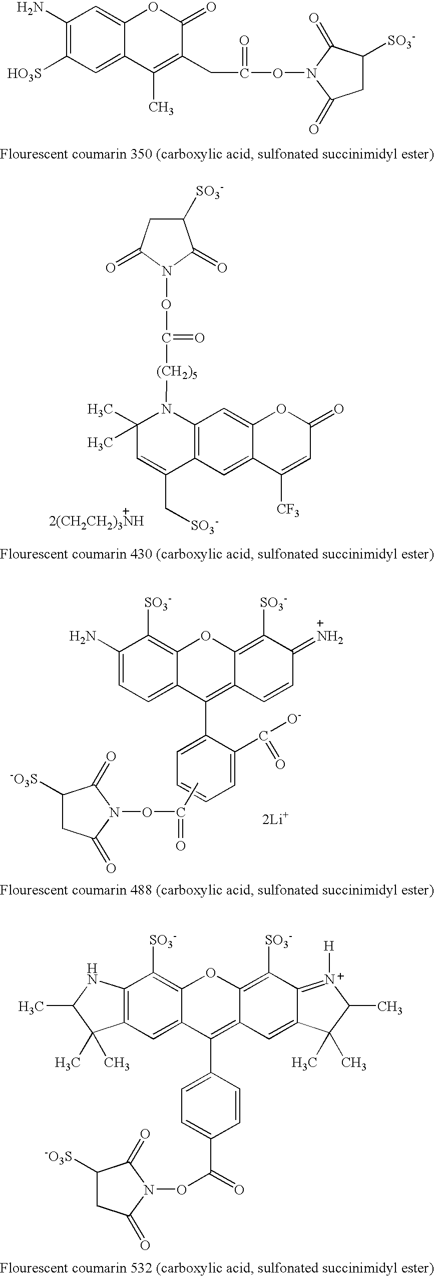

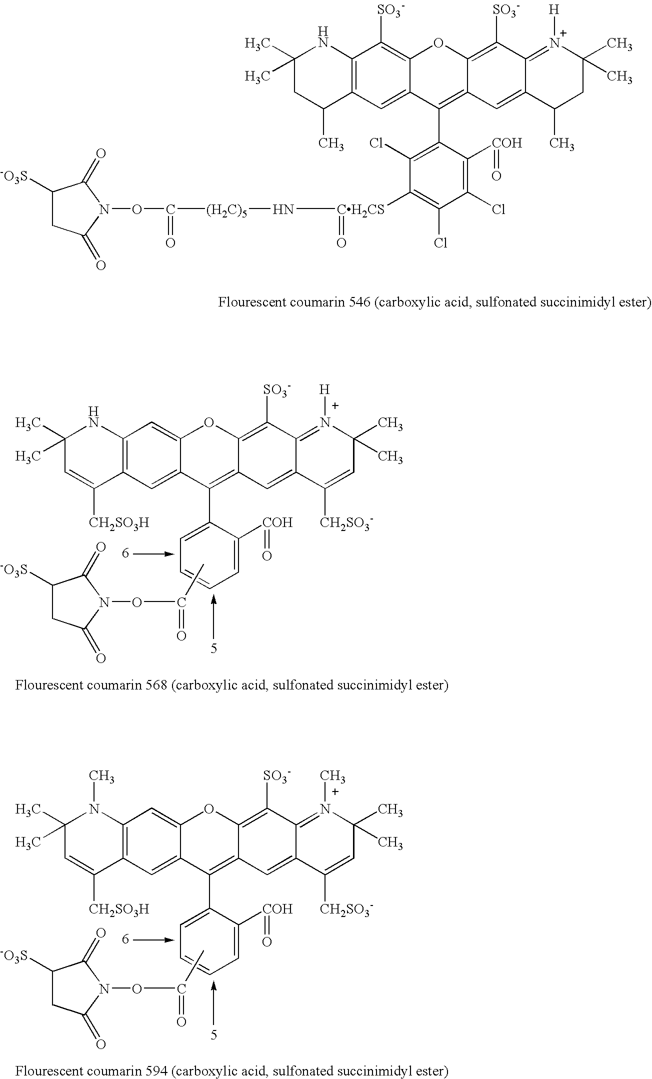

Certain preferred labeling agents include NHS-SS-biotin (EZ-Link™ NHS-SS-Biotin, Cat. No. 21331, Pierce, Rockford, Ill.), wherein the biotin or the disulfide bond may be considered the marking moiety and NHS is the protein binding moiety, and/or any of the above fluorescent coumarins shown above, such as coumarin 488 carboxylic acid, succinimidyl ester, dilithium salt, available through Molecular Probes, Inc. as Alexa Fluor®488 (Cat. No. A-10235, Molecular Probes, Eugene, Oreg.), wherein the fluorescent coumarin is the marking moiety and the succinimidyl ester is the protein binding moiety.

Another exemplary labeling agent is an Isotope Coded Tag (ICT). An ICT comprises a marking moiety that may carry one or more stable isotopes, preferably deuterium. Another variant is an Isotope Coded Affinity Tag (ICAT), which additionally comprises a marker for affinity purification, such as biotin. Exemplary ICAT labeling agents may be found in Aebersold et al. (Nature Biotechnology (1999) 17: 994-999).

Methods for labeling membrane surface proteins generally comprise contacting closed membrane structures with a labeling agent for sufficient time to allow stable interactions to form between the labeling agent and membrane surface proteins. In many embodiments, cells are contacted with labeling agent for sufficient time, lysed and the labeled proteins are analyzed. In certain embodiments, the marking moiety is suitable for affinity purification, and labeled proteins may be separated from unlabeled proteins by affinity purification.

In other embodiments, the marking moiety is not suitable for affinity purification but is easily detectable, for example a fluorescent marking moiety. In such cases, cell surface proteins are enriched through any of various methods for enriching membranes. Such methods are, in view of this specification, generally known to one of skill in the art. Typically, membranes and the associated proteins are enriched by a separation method that takes advantage of the difference in density between membranes and other cellular components. For example, gradient centrifugation will yield a fraction of membrane material largely separated from other, non-membrane-associated, cellular components. Other separation methods may take advantage of the poor solubility of membranes in aqueous solutions. For example, insoluble membranes may be separated from soluble components by high-speed centrifugation. Membranes isolated in this fashion will comprise both labeled and unlabeled proteins, but the detectable marking moiety permits the identification of those proteins that are labeled with the labeling agent.

Cells to be labeled may be cultured cells as well as cells obtained from a subject. In preferred embodiments, cells are eukaryotic cells with intact membranes, and in some embodiments, the cells are viable. Preferably, cells are stripped of extracellular matrix prior to labeling so as to tag only those proteins that remain associated with the membrane after removal of the extracellular matrix. An exemplary procedure for removing extracellular matrix from adherent cultured cells comprises detaching cells using a physiological salt buffer (eg. phosphate buffered saline—“PBS”) and a divalent ion chelator (eg. EDTA) solution. The chelating agent causes depolymerization of extracellular matrix proteins, which are subsequently washed away by one or more salt buffer washes. Thus, only proteins that are associated with the cell surface will remain and be labeled in subsequent steps. Similar methods may be employed to remove the extracellular matrix from cells obtained from a subject.

In certain preferred embodiments, the labeling reaction is performed at a temperature cold enough to minimize membrane protein turnover. Preferred temperatures range from about 1 degree C. to 10 degrees C., and most preferably the temperature is about 4 degrees C. An exemplary buffer for labeling with a succinimide-based protein binding moiety is PBS/CM (PBS with 1.3 mM CaCl₂, 1 mM MgCl₂). The binding reaction between the labeling agent and the proteins must often be quenched. For example, a labeling agent that covalently binds to amines can be quenched with a compound containing amines, eg. glycine or Tris. Quenching may also be accomplished by lowering the pH by, for example, adding ammonium chloride, or by a combination of pH lowering and the addition of primary amines. Quenching is typically followed by a wash in a physiological salt buffer and then transfer into a solubilization buffer. Solubilization buffers typically comprise buffering agents at pH 6-8, divalent cations, salts and a non-ionic detergent. Exemplary detergents include Triton X-100 and, most preferably ASB-14. An exemplary solubilization buffer contains 50 mM Tris-HCl, pH7.6, 150 mM NaCl, 10% glycerol, 2% ASB14, 5 mM EDTA, 1 mM EGTA, 1.5 mM MgCl2, and protease inhibitors. Solubilization is usually carried out at a cool temperature to minimize damage to the proteins. After solubilization, the labeled proteins are separated from the unlabeled proteins.

In certain embodiments, biotin is used as the marking moiety of the labeling agent. The resultant labeled proteins are therefore biotinylated. Such proteins are preferably affinity purified by contacting them with a biotin-binding substrate such as avidin-sepharose beads. After the binding reaction, unbound proteins are removed by washing. Suitable wash buffers are, in view of this specification, known to those of skill in the art. An exemplary buffer comprises 20 mM Tris-HCl, pH7.6, 300 mM NaCl, 10% glycerol, 0.1% Triton X-100, 0.1% SDS, 1 mM EDTA, 1 mM EGTA, 1.5 mM MgCl2, and protease inhibitors. Release of biotinylated proteins from avidin beads can be difficult. One method is to use a reversible connection between the protein binding moiety and the biotin. Preferred reversible connections are disulfide bonds which may be broken by reduction with an appropriate reducing agent. Application of the reducing reagent may result in a dramatic reduction in pH which, depending on the downstream use for the protein preparation, may be undesirable. Preferably, the reduction is accomplished using TCEP-HCl (Cat. No. 580560, Calbiochem), due to its superior stability and effectiveness over a wide pH range (1.5-8.5). The reducing solution is then strongly buffered, for example by addition of greater than 25 mM Tris base, and most preferably by addition of approximately 50 mM Tris base. Most preferred reducing solutions will have sufficient buffering material added to have a pH in the range of 6.5 to 8, most preferably about 7.5. An exemplary reducing solution comprises 50 mM Tris base, 20 mM TCEP-HCl, 20 mM NaOH and most preferably further includes a cocktail of protease inhibitors and/or roughly 150 mM NaCl. Reduction may be performed at essentially any temperature that is favorable for recovery of protein, and in certain embodiments, reduction is performed at a temperature ranging from 20 to 30 degrees C., preferably at room temperature. After incubation, labeled proteins substantially free of unlabeled proteins are available for further analysis. This method may result in the production of free thiols that, as described above, can be used to label proteins with a second labeling agent that reacts with thiols. This procedure may, depending on the method in which it is employed, provide a number of advantages. For example, in the detection of relatively low abundance membrane proteins, after enrichment of membrane proteins according to the biotin/avidin method described above, the free thiols may be reacted with a radioactive labeling agent. These labeled membrane proteins can then be identified using gel electrophoresis and the time of exposure to a system for detecting radiation (such as a film or a Phosphoimager, available from Amersham Biosciences) such that low abundance proteins can be detected. Alternatively a fluorescent label may be used for the second labeling to allow detection by fluorescent systems. In addition, if distinguishable fluorescent labels are used for different preparations of labeled membrane surface proteins, then a differential display of membrane proteins can be achieved. Fluorescent detection systems may be coupled with chromatography as well as gels.

As described above, a variety of labeling agents may be designed to incorporate a reversible bond such as a disulfide bond. The reduction protocol described above for use with a biotin/avidin system may also be used with other labeling agents comprising a disulfide bond, and the reducing conditions described therein may be used to generate the free thiols regardless of the moiety attached thereto. For example, the reducing conditions may be used to generate free thiols from any of the labeling agents comprising a PBA- or SHA-type group and a disulfide.

In a further embodiment, a lectin is used as the protein-binding moiety. Lectins may easily be modified with any appropriate fluorescent marking moiety.

In an additional embodiment, the labeling agent comprises a fluorescent coumarin as the marking agent and succinimidyl ester as the protein binding moiety. The succinimidyl ester binds covalently to primary amines and the steps of the labeling process are essentially as described above. However, fluorescent coumarins are not easily used for affinity purification. Accordingly, the labeled cell surface proteins may be substantially enriched by using membrane enrichment methods described above. Alternatively, labeled proteins may be directly resolved, for example by two-dimensional (2D) electrophoresis, and cell surface proteins are distinguished from other proteins by the fluorescent label.

The invention provides for differential display methods that permit direct comparison of two or more samples. In general, a first sample is reacted with a first labeling agent and a second sample is reacted with a second labeling agent. The first and second labeling agent are typically identical except for having a detectably different marking moiety. In a preferred embodiment, two samples are treated with lectins modified with different fluorophores. The samples are then mixed and resolved. In certain embodiments, resolution is accomplished by electrophoresis and preferably two-dimensional electrophoresis. All of the proteins migrate to the appropriate position on the gel, and the comparative amount of each protein in each sample is measured by reading the respective fluorescence signals. In an alternative embodiment, fluorescent non-lectin labeling agents are used. In yet another embodiment, the labeling agents comprise ICT or ICAT moieties. For example, the first sample is labeled with a “light” or non-deuterated ICT or ICAT, while the second sample is labeled with a “heavy” or deuterated ICT or ICAT. The differentially labeled samples are mixed and subjected to mass spectrometry. MS is capable of distinguishing the “heavy” labeled proteins from the “light” labeled proteins and provides a direct comparison of the amount of each labeled protein present in each sample. As discussed above, many different fluorophores, and potentially many distinguishable ICT or ICAT moieties are available, and it is anticipated that the methods described herein may be used with more than two samples, so long as each sample is labeled with a distinguishable marker. For example, three samples may be differentially labeled with red, green and blue fluorescing moieties, mixed and analyzed to provide a differential display of the relative membrane surface protein abundance in each sample.

4. Methods of Processing and Identifying Membrane Proteins

Having obtained an enriched preparation of membrane surface proteins, it is generally desirable to identify and characterize the proteins present. In certain embodiments, methods of the invention include the identification of proteins present in the preparation. In preferred embodiments, a plurality of proteins are identified, and it is particularly preferable to identify more than 10, more than 20, 25, 30, 50, 100 or 1000 proteins in a preparation. It is also desirable to characterize other aspects of each protein, such as abundance, the presence of one or more post-translational modifications, and membrane topology.

In certain aspects, the invention provides methods of generating profiles of membrane surface proteins. In general, a profile of membrane surface proteins is obtained by combining a step of selectively labeling membrane surface proteins with a step of identifying labeled proteins. Preferred profiles will include information about the identity and amount of membrane surface proteins present in a sample. Profiles may be generated for a number of different samples, possibly representing a range of tissue types and clinical states. A profile may be compared against one or more other profiles. Such comparisons may be useful for indicating changes in protein levels, modifications, etc. In addition, such comparisons may be used to characterize a sample. For example, a profile from a possible cancer sample may be compared against a range of cancerous and non-cancerous profiles to determine whether the sampled material is indeed cancerous.

In view of this specification, many techniques for identifying and/or characterizing proteins of the subject preparations are available to one of skill in the art. Certain methodologies require preparative steps, such as, for example, resolution of the complex mixture of proteins into simpler mixtures or substantially pure proteins. Other methodologies may be used without such preparative steps. While not intended to be limiting, several preferred methods of analysis are presented herein. Such methods may be combined in various ways that, in view of this specification, will be appreciated by one of skill in the art.

Gel Electrophoresis

Gel electrophoresis of proteins is a common methodology that may provide many different forms of information, including protein size, isoelectric point and abundance. In addition, gel electrophoresis is a powerful method for the resolution of complex protein mixtures into bands or spots of reduced complexity. One dimensional electrophoretic methods include, but are not limited to, one-dimensional SDS-PAGE, isoelectric focusing, one-dimensional non-denaturing gel electrophoresis and 2D gel electrophoresis. 2D gel electrophoresis involves one dimension of isoelectric focusing and another dimension of SDS-PAGE. Proteins resolved by gel electrophoresis may be used for further analysis, if desired. Proteins may be eluted or otherwise obtained from the gel by a variety of methods including, for example by cutting the appropriate portion of the gel, optionally followed by electroelution, or alternatively by electroblotting onto a membrane such as nitrocellulose or polyvinylfluoride. Proteins so processed may then be used in a variety of analytic methods, including but not limited to, antibody analysis (eg. Western blot, ELISA, protein array), Edman degradation, mass spectrometry, etc.

Chromatography

Proteins may be resolved into simpler mixtures or to substantial purity by a variety of chromatography methods known in the art. Such chromatography methods may include, for example, anion exchange, cation exchange, hydrophobic interaction, reverse phase, size exclusion, hydroxylapatite etc. In addition, a variety of affinity chromatography methods may be employed, depending on the particular proteins of interest. Chromatography methods may be employed in series or performed repeatedly to obtain higher degrees of resolution. Chromatography is not only a preparative tool. Many types of chromatography provide information about the proteins. For example, size exclusion chromatography can be used to obtain molecular weights of native proteins in both reducing and non-reducing conditions. Ion exchange columns provide information regarding the pI of the subject proteins.

Mass Spectrometry

With the extensive availability of protein sequence information, mass spectrometry (MS) may be employed for rapid identification of proteins present in cell surface protein preparations. Mass spectrometry may also be useful for determining post-translational modifications and membrane topology.

Sample Preparation for Mass Spectrometry

If proteins are first resolved by gel electrophoresis, certain preparative steps are preferred. In order to facilitate the identification of proteins by MS, bands containing one or more protein species are excised from the gel, digested into polypeptides by treatment in situ with a protease such as trypsin, and transferred into solutions and concentrations compatible with MS analysis. Techniques for the in-gel processing of proteins have been refined into standardized protocols. The so-called “in-gel digestion” approach has been developed for the enzymatic fragmentation of proteins embedded in gel pieces, and the extraction of the resulting peptides (Wilm et al. (1996) Nature 379: 466-9). Sequencing-grade modified trypsin has been the enzyme of choice for high-throughput identification of proteins. In one exemplary method, a band of interest is excised from the gel, and subjected to reduction and alkylation to break the cysteine bridges and prevent them from reforming. After equilibration with the corresponding buffer the gel pieces are swelled in a solution of trypsin, allowing the enzyme to enter into the gel. The digestion is allowed to proceed at 37° C., generally overnight. The resulting peptides are extracted and prepared for MS analysis.

Mass Spectrometers for Protein Identification

Typically, a mass spectrometer consists of at least three components: an ionization device, a mass separator, and a detector. Mass spectrometry is a very powerful separation technique for separating and identifying molecules that are charged in the gas phase. Mass spectrometers are generally only able to separate either positively or negatively charged analytes at a time. The term ionization is misleading, because most mass spectrometers do not perform the ionization of molecules per se. Instead, the term ionization relates to the transfer to gas phase of analytes, while maintaining their charge, and/or acquiring a charge from the sample environment, typically in the form of proton. The study of peptides and proteins is predominantly dominated by two sample ionization techniques: matrix-assisted laser desorption ionization (MALDI) (Aebersold et al. (1993) Curr Opin Biotechnol 4: 412-9; Arnott et al. (1993) Clin Chem 39: 2005-10; Hillenkamp et al. (1991) Anal Chem 63: 1193A-1203A). and electrospray ionization (ESI) (Fenn et al. (1990) Mass Spectrometry Reviews 9: 37).

MALDI Mass Spectrometers, Peptides and Proteins Analysis

MALDI ionization is a technique in which samples of interest, in this case peptides and proteins, are co-crystallized with an acidified matrix (Nelson et al. (1994) Rapid Commun Mass Spectrom 8: 627-31.). The matrix is a small molecule, which absorbs at a specific wavelength, generally in the ultraviolet (UV) range and dissipates the absorbed energy thermally. Typically, a pulse laser beam is used to rapidly (few ns) transfer energy to the matrix. This rapid transfer of energy causes the matrix to rapidly dissociate from the surface generating a plume of matrix and the co-crystallized analytes into the gas phase. It is not clear if the analytes acquire their charge during the desorption process or after entering the gas plume of molecules by interacting with the matrix molecules. However, the end result is a small pocket of charged analytes that are present in the gas phase. To date, MALDI has been predominantly coupled in-line with time of flight (TOF) mass spectrometers. The function of a time of flight mass spectrometer is to measure the time that analytes take to flight across a fixed path length (the TOF tube or chamber). The charged analytes present in the plume are therefore transferred to the TOF tube after an appropriate time delay. In order to move the analytes into the TOF tube, a high voltage is applied to the MALDI plate generating a strong electric field between the plates and the entrance of the TOF chamber. Smaller analytes will reach the entrance of the chamber more rapidly than larger analytes (i.e. constant kinetic energy applied, generating different velocity for the analytes). Once in flight, the analytes are in a field-free region and separate along the tube while moving toward the detector. Again, analytes of lesser mass move along the tube faster and reach the detector prior to analytes of greater mass. The detector is in tune with the laser shots and time delay, and measures the peptide and protein ions as they arrive over time. When the mass range is calibrated by using standards of known mass and charge, the time of flight for a given ion can be converted to masses. The end result is a spectrum comparing observed intensity versus ion (protein or polypeptide) mass.

MALDI-TOF MS is easily performed with modern mass spectrometers. Typically the samples of interest, in this case peptides or proteins, are mixed with a matrix mixture and successively spotted onto a polished stainless steel plate (MALDI plate). Commercially available MALDI plates can hold 96 samples per plate. The MALDI plate is then installed into the vacuum chamber of a MALDI mass spectrometer. The pulsed laser is then activated and the time of flight acquisition triggered as previously described. An MS spectrum containing the masses mass to charge ratio of the peptides/proteins is then generated. The charge of molecules ionized by MALDI is typically 1.

Recently, the MALDI ion source technology has also been coupled with a hybrid orthogonal mass spectrometer. In this design the MALDI ionization approach is, but for minor modifications, essentially as described above. However, the TOF detector is replaced with an orthogonal mass spectrometer (e.g. Q-Star by PE-Sciex), which consists of a quadrupole followed by a collision cell and a pulsed perpendicular TOF MS. The hybrid instrument (MALDI-Q-Star) has the advantages of high resolution mapping of the peptide masses contained in a peptide mixture, and the option of efficient fragmentation of selected peptides by collision induced dissociation. These fragmentation patterns contain information related to the amino acid sequence of the peptides.

ESI Mass Spectrometers, Peptides and Protein Analysis

Electrospray ionization is also widely utilized to introduce protein and peptide mixtures to mass spectrometers. Electrospray ionization (ESI) allows the transfer of analytes from a liquid phase to the gas phase at atmospheric pressure. The ionization process is achieved by applying an electric field between the tip of a small tube and the entrance of a mass spectrometer. The electric field induces the charged liquid at the end of the tip to form a cone, called a Taylor cone that minimizes the charge/surface ratio. Droplets are liberated from the end of the cone, and travel towards the mass spectrometer entrance. The liberated droplets go through a repetitive process of solvent evaporation from the droplets and fragmentation of the droplets into smaller droplets. This process leads to a large number of droplets of vanishing size until the solvent has disappeared and the charged analytes are in the gas phase. Moreover, while the droplets are shrinking, the pH decreases causing protonation of the analytes. Therefore, it is common to obtain multiply charged analytes by ESI when dealing with trypsinized proteins.

Typically, electrospray ionization is used in conjunction with triple quadrupole, ion tap, or hybrid quadrupole-time-of-flight mass spectrometers (Patterson et al. (1995) Electrophoresis 16: 1791-814). Electrospray ionization has significant advantage over MALDI in terms of ease of coupling to separation techniques such as HPLC, LC and CE. ESI can also be used for the continuous infusion of samples. Furthermore, the tendency to provide multiply charged peptides from tryptic digests, in conjunction with collision-induced dissociation allows the generation of enhanced MS/MS spectra over what has been achieved with either conventional MALDI-TOF, or the hybrid MALDI-Q-Star instrument.

Electrospray ionization and the MALDI-Q-Star instruments both rely on collision-induced dissociation to generate fragmentation patterns (MS/MS spectra) related to a selected peptide amino acid sequence. Typically the generation of MS/MS spectra requires two independent experiments. In the first pass, a mixture of peptides (a tryptic digest) are separated according to mass-to-charge (m/z) ratio by the mass spectrometer and a list of the most intense peptide peaks is established. In the second pass, the instrument is adjusted such that only a specific m/z species (identified during the first-pass analysis), presumably a unique peptide ion, is allowed to enter the mass spectrometer. These ions are directed into a collision cell and their kinetic energy is increased. In the collision cell the ions collide with inert gas molecules with sufficient kinetic energy to break peptide bonds. This process is termed collision-induced dissociation, CID, and generates both charged and neutral fragments derived from the same ‘parent’ ion. Finally, the newly generated charged fragments are separated by the mass spectrometer according to their m/z creating the MS/MS spectrum. By application of appropriate collision energy, the fragmentation occurs predominantly at the peptide bonds and a ladder of fragments is generated. The difference in mass between certain peaks corresponds to the loss of a single amino acid. The sequence of the peptide can then be reconstituted by a ladder-walk done by measuring the mass difference between successive masses for specific types of ions (i.e. y or b series ions).

The peptide masses are typically accurately measured using a MALDI-TOF or a MALDI-Q-Star mass spectrometer down to the low ppm (parts per million) precision level. The ensemble of the peptide masses observed in a tryptic digests can be used to search protein/DNA databases in a method often called peptide mass fingerprinting (Clauser et al. (1995) Proc Natl Acad Sci USA 92: 5072-6; Cottrell (1994) Pept Res 7: 115-124; Pappin (1997) Methods Mol Biol 64: 165-73). In this approach protein entries in the databases are ranked according to the number of peptide masses that match to their predicted trypsin digestion pattern. Commercially available software provides a scoring scheme based on the size of the databases, the number of matching peptides, and the different peptides. Depending on the number of peptides observed, the accuracy of the measurement, and the size of the genome of the particular species, unambiguous identification can be obtained.

MS/MS spectra are a second set of information that can be used to identify a protein. The MS/MS spectra contain the fragmentation pattern related to the amino acid sequence of specific peptides. The analysis of MS/MS spectra is typically more intensive. The approaches that are in used for the interpretation of these spectra can be classified into three subgroups according to the level of user intervention required.

In the first subgroup no interpretation of the spectra is required. The information contained in the spectra is directly correlated with protein/DNA sequence information contained in databases. Different algorithms have been developed for this specific task. These algorithms automatically search uninterpreted MS/MS spectra against protein and DNA databases and some are freely available (for non-commercial entities) and can be accessed over the Web. Mascot by Matrix Sciences (www.matrixscience.com), and ProteinProspector from UCSF (http://prospector.ucsf.edu) are the most commonly used web-based MS/MS search engines. The identification of the protein is typically unambiguous through the number of peptides that matches to the same protein. Another algorithm that is popular is “Sequest” (Eng et al. (1994) J. Am. Soc. Mass Spectrom. 5: 976-989; Yates et al. (1995) Anal Chem 67: 1426-36; Yates et al. (1998) Peptide sequencing by tandem mass spectrometry, p. 529-538, Cell Biology: A Laboratory Handbook, vol. 4. Academic Press, San Diego). For every MS/MS spectra submitted this algorithm searches protein/DNA databases for the top 500 isobaric peptides and the corresponding predicted spectra are generated. The predicted spectra are rapidly matched against the measured spectra by multiplication in the frequency domain using a fast-Fourier transformation. Correlation parameters, which indicate the quality of the match between predicted and measured spectra, are then deduced. A high cross-correlation indicates a good match with the measured spectrum. Although protein identification has been performed with as little as one peptide using this algorithm, unambiguous identification of the provenance of a protein is often achieved by the multitude of peptides that matches to the same entry in a database. The Sequest algorithm is computing intensive, and for high-throughput demand can rapidly paralyze a dual-CPU server. The slow nature of Sequest is due to its attempt to find the best matching 500 isobaric peptides. The larger the database being repeatedly scanned to compile this list, the longer this function takes. An improved version of the software, called Turbo-Sequest, predigests and orders the databases resulting in greatly improved searching times.

The approaches in the second subgroup all involve the partial interpretation of the MS/MS spectra, and therefore require human intervention. The dominant approach, often called “sequence-tag” (Mann et al. (1994) Anal Chem 66: 4390-9; Patterson et al. (1996) Electrophoresis 17: 877-91; Wilkins et al. (1996) Biochem Biophys Res Commun 221: 609-13), consists of reading the mass spacing between a few specific fragments in a MS/MS spectrum and to generate a short section (tag) of the peptide sequence. Using this tag and the residual mass information, the provenance of the peptide can be ascertained by comparison with sequence and calculated masses obtained from protein databases for isobaric peptides. Every MS/MS spectrum requires the generation of a tag followed by database searching. Unambiguous identification of the protein is established by the multitude of peptides that match to the same protein. Over the years, different variations on this theme have been developed to perform database searching using sequence tags. The main limitation of the “sequence-tag” approach in large-scale proteomics efforts is the labor and expertise required to manually generate the required partial interpretations of the MS/MS spectra. Attempts to automate the generation of sequence tags are underway to solve this problem.

The last sub-group, called de novo sequencing of proteins (Shevchenko et al. (1997) Rapid Commun Mass Spectrom 11: 1015-24; Papayannopoulos et al. (1995) Mass Spect. Rev. 14: 49-73), is often used as a last resource when no matching information are available in databases and the quality of the MS/MS spectra is good. The MS/MS spectra of peptides contain ladder-type information, which, in principle indicates their amino acid sequence. Experienced mass spectrometrists can manually extract the peptide sequence from the CID spectra (de novo sequencing).

Depending on the quality of the data and the complexity of the species under study, a single confident match between a peptide MS/MS spectrum and a protein sequence entry can be enough to identify a protein, or a family of proteins. The required sequence coverage for unambiguous identification increases for homologous proteins, when the peptide identified is not unique to a protein, when dealing with databases of poor fidelity and/or partial coverage, and to access SNP databases. Clearly, every subsequent peptide MS/MS that is matched to the same protein further increases the confidence level of the identification.

The end result of each of these MS-based approaches is the delivery of the identity of the proteins presented for analysis or the partial amino acid sequence of novel proteins.

Antibody-Related Methods

Antibodies are powerful tools for protein identification, quantitation and isolation. Following gel electrophoresis, Western blotting methods may be performed using one or more antibodies to identify and, if desired, quantify a number of different proteins present in a preparation. Enzyme-linked immunosorbent assays (ELISAs) may also be performed to quantify protein levels in a sample. Parallel ELISAs using a range of different antibodies may be performed in a high-throughput method to rapidly obtain quantitative information about many different proteins in a sample. Antibodies may also be used as a part of a protein identification array (discussed below).

Protein purification can also be achieved using antibodies. For example, antibodies may be conjugated to a matrix and used for immunoaffinity chromatography. Purification can also be achieved by immunoprecipitation. Typically a protein mixture is contacted with one or more antibodies, and then the antibody-associated proteins are precipitated by addition of beads coated with an antibody-specific binding agent, such as protein A. Antibodies may also be tagged with, for example, a biotin molecule, so that precipitation can be achieved using a streptavidin matrix.

It is understood that antibodies come in a variety of forms including single chain antibodies, polyclonal, monoclonal, Fab fragments, etc.

Protein Identification Arrays

The identity, abundance and even post-translational modification state of proteins in a complex mixture can be determined using any of a variety of protein identification arrays (WO 00/04389; WO 00/04382; WO 00/04390). In general, a protein identification array is an ordered array of protein capture agents, wherein each protein capture agent is capable of binding to a particular protein. Protein capture agents may be specific to a particular protein or to certain epitopes, including post-translational modifications. The interaction of a protein capture agents with the corresponding protein(s) may be sensitive or insensitive to post-translational modifications. In general, protein capture agents bind to their binding partners specifically and with a dissociation constant (K_{D}) less than 10⁻⁶. Protein capture agents will typically be a biological molecule such as a polypeptide or a polynucleotide (including standard nucleic acids and artificial nucleic acid analogs with altered bases and/or altered backbones, including peptide nucleic acids, locked nucleic acids, linked nucleic acids, mannitol, hexitol, glucitol etc. nucleic acids). For example, antibodies are highly suitable protein capture agents.

Protein capture agents may be organized into arrays through a variety of methods. In general, arrays can be sorted into three types: (1) arrays wherein the protein capture agents are distributed, typically in solution, in a plurality of wells; (2) arrays wherein the protein capture agents are affixed to a plurality of positions on a solid substrate; (3) arrays wherein the protein capture agents are distributed as discrete spots within a gelatinous or porous substrate. In each case, the array is organized such that the protein(s) expected to bind to each position on the array are known. The smaller each position of the array, the greater the number of protein capture agents that can be included within an area Miniaturization is beneficial because it reduces the sample size required to obtain a readable signal, reduces the amount of each protein capture agent needed, and permits smaller instruments for the production and analysis of the arrays. In an example of array type (2), a silicon wafer is coated with a grid of gold and titanium. An amino-reactive compound (eg. 11,11′-dithiobis(succinimidylundecanoate) is applied to the gold surfaces and then used to immobilize antibodies spotted onto the array.

The procedure to analyze a complex mixture of proteins is, in general, as follows. A mixture of proteins is applied to the protein identification array. If a protein of the mixture can be bound by a protein capture agent of the array, the protein will localize to that particular position on the array. The array is designed such that it is known which proteins will bind to which positions on the array. Therefore, much as with nucleic acid identification arrays, each protein can be identified by the position on the array that it binds to. Proteins on the array can be measured by a variety of methods. Generally, the proteins will be labeled prior to application to the array. Labels may include any of those discussed herein. The amount of protein present at each position of the array may be measured by measuring the presence of the label.

Protein identification arrays may be comprehensive, encompassing as many proteins and protein variants as possible, or the array may be selective, representing only a subset of proteins or protein types.

Edman Degradation

Protein identification may be accomplished by any of a variety of sequencing methods. For example, the most commonly used sequencing methods include amino-terminal sequencing using the Edman degradation method and mass spectroscopy (see above). In general, Edman degradation is useful for obtaining the amino-terminal sequence of a purified polypeptide. Internal sequence of the polypeptide may be obtained by fragmenting the polypeptide (eg. through proteolysis), thereby generating internal fragments with free amino-termini. Edman degradation is most effective within the 15-30 amino acids most proximal to the amino terminus. With the availability of extensive databases of nucleic acid and protein sequences, it is usually unnecessary to obtain a complete protein sequence in order to make an unambiguous identification. One or more fragmentary sequences may be compared against sequence databases to identify matches. Typically 15-20 amino acids will be sufficient to make an unambiguous identification, particularly when combined with information such as predicted molecular weight and species of origin.

In an exemplary embodiment, a protein is attached to a solid support such as a chemically modified glass disk or a porous polyvinylidene fluoride membrane in the reaction cartridge. It is then coupled to phenylisothiocyanate (PITC) at pH 8 and 45° C. The free N-terminal amino group reacts with the carbon of the isothiocyanate group to give the phenylthiocarbamyl (PTC) derivative of the peptide. The next step is cleavage of the PTC derivative using anhydrous trifluoroacetic acid to give the anilinothiozolinone (ATZ) derivative of the N-terminal amino acid, and the peptide with one fewer amino acid, which is free to undergo further couplings and cleavages. The ATZ residue is then filtered into the conversion flask, where it is converted to the phenylthiohydantoin (PTH) amino acid. This is a two step process. First, the ATZ derivative is hydrolyzed under aqueous, acidic conditions to give the PTC amino acid. The acid then cyclizes to give the stable PTH derivative. These derivatives are then injected into an high pressure liquid chromatography (HPLC) column where its retention time is compared with that of known PTH amino acid standards. The reaction is then repeated with the remaining C-terminus of the original peptide. Thus, each round of the Edman reaction identifies one further amino acid residue in a protein.

X-Ray Diffraction Crystallography

In an embodiment, a protein sequence and structure may be studied using X-ray diffraction crystallography. In this method, a crystal of the protein is prepared. Methods of solubilizing and growing crystals of membrane proteins are described, for example, in U.S. Pat. No. 6,172,262 to McQuade et al, and in U.S. Pat. No. 6,174,365 to Sanjoh. X-rays are directed onto the crystal to produce diffracted beams, which are subsequently detected by film or various electronic detectors. The pattern of diffraction is determined in part by the atomic structures on which the incident X-rays impinge and from which they diffract. In a crystal, these atomic structures are regularly ordered, so that the diffracted X-rays form regular patterns of interference. A particular diffraction pattern may therefore be associated with a particular arrangement of atoms. Thus, the appearance of a given diffraction pattern may suggest to one of ordinary skill in the art that the crystal being studied comprises the corresponding atomic structure.

Each atom in a crystal scatters x-rays in all directions, and only those that positively interfere with one another, according to Bragg's law, give rise to diffracted beams that can be recorded as a distinct diffraction spot above background. Each diffraction spot is the result of interference of all x-rays with the same diffraction angle emerging from all atoms. For example, for the protein crystal of myoglobin, each of the about 20,000 diffracted beams that have been measured contain scattered x-rays from each of the around 1500 atoms in the molecule.

Integral membrane proteins have traditionally been more difficult to obtain crystal structures from, but recent developments have made this increasingly possible and rapid. (Abramson et al. (1999). “Crystallization of membrane proteins” in “Crystallization of proteins: techniques, strategies and tips, a laboratory manual”. (Edited by Bergfors, T.), International University Line, La Jolla, Calif. 199-210; Abramson, et al. (2000) Nat. Str. Biol. 7 (10); Byrne et al. (2000) Biochim. Biophys. Acta. 1459, 449-455; Iwata et al. (1995) Nature 376, 660-669; Iwata et al. (1998) Science 281, 64-71; Michel et al (1982) J. Mol. Biol. 158, 567-572; Ostermeier, et al. (1995) Nature Str. Biol. 2, 842-846; Landau et al. (1996) Proc. Natl. Acad. Sci. USA. 93, 14532-14535).

Nuclear Magnetic Resonance

In an embodiment, NMR may be used to analyze the structure of membrane proteins. Briefly, the technique involves placing the material to be examined (usually in a suitable solvent) in a powerful magnetic field and irradiating it with radio frequency (rf) electromagnetic radiation. The nuclei of the various atoms will align themselves with the magnetic field until energized by the rf radiation. They then absorb this resonant energy and re-radiate it at a frequency dependent on i) the type of nucleus and ii) its atomic environment. Moreover, resonant energy can be passed from one nucleus to another, either through bonds or through three-dimensional space, thus giving information about the environment of a particular nucleus and nuclei in its vicinity.

Certain atoms are particularly well suited to analysis using NMR. For example, most early NMR work detected resonance energy from ¹H atoms. Over the past few years, labeling proteins with ¹⁵N and ¹⁵N/¹³C has raised the analytical molecular size limit to approximately 15 kiloDaltons (kD) and 40 kD, respectively. More recently, partial deuteration of the protein in addition to ¹³C- and ¹⁵N-labeling has increased the size of proteins and protein complexes still further, to approximately 60-70 kD. See Shan et al., J. Am. Chem. Soc., 118: 6570-6579 (1996) and references cited therein.

Membrane Topology

The methods described herein may be used for determination of membrane topology. Labeling agent will bind only to those portions of protein that are exposed to the environment external to the membrane structure. Accordingly, the position of labeling agent on each protein creates a record of which portions of the protein are exposed on the external face of the membrane. The position of label on each protein may be determined by, for example mass spectrometry analysis of digested, labeled proteins. Each fragment of a protein may be identified as labeled or unlabeled and assigned as an external or internal fragment, respectively. To be so assigned, a fragment should have at least one amino acid that can react with the labeling reagent. Any fragment unable to react with labeling agent will of course not be labeled, and the therefore can not be assigned topologically. It is anticipated that this methodology would permit high-throughput determination of membrane topology by rapid analysis of fragmented, labeled proteins.

Delivery Systems for High-Throughput Identification

Each of the above-described methods for identifying the sequence and/or structure of membrane proteins may be employed in a system designed for high-throughput identification. Techniques such as Liquid chromatography, Gas chromatography, Gel permeation chromatography, Size exclusion chromatography, Solid phase extraction, Capillary electrophoresis, and Capillary electrochromatography are all well-known methods for preparing and delivering analytical samples to XRC, NMR, MS, and Edman degradation devices.

5. Diagnostic Assays and Cell Surface Markers

In certain aspects, the invention provides methods for comparing the biological states of cells by comparing the membrane surface protein profiles from different cell samples. In general, a comparative method may comprise treating a first sample with a labeling agent and treating a second sample with a labeling agent. Each of the labeled samples is then processed to produce a preparation of labeled cell surface proteins. A plurality of cell surface proteins from each preparation are analyzed to identify the proteins, and, preferably, to obtain quantitative and/or qualitative information about each analyzed protein. The information obtained about each surface protein preparation forms a profile, and the profiles from different samples may be compared to identify differences and similarities between the samples.

In certain embodiments, profiles may be treated as fingerprints that are indicative, as a whole, of a particular sample type and its associated biological state. As an illustrative example, surface protein profiles from healthy tissues and cancerous tissues may be obtained and recorded. A sample of unknown health status may then be used to prepare a surface protein profile, and this profile is compared against previously obtained profiles to determine whether the sample more closely matches healthy or cancerous tissue. In preferred embodiments, statistical methods are used to identify characteristics of surface protein profiles that are most indicative of particular biological states. For example, a subset of surface proteins may be particularly associated with a cancerous state. In this manner, methods of the invention may be used to identify cell surface markers that are diagnostic of particular biological states.

It is expected that essentially any two cells with different biological properties will evince differences in cell surface protein composition. Accordingly, methods of the invention will be useful in profiling and/or identifying cell surface markers for essentially any biological property of interest.

Exemplary biological states are presented herein solely for the purposes of illustration.

Cancer

Cancers, or neoplasms, develop through a series of stages including the initial formation of a modified tumor cell, formation of a localized tumor mass, development of invasive properties, and metastasis to distal sites. While the progression and genetic abnormalities of each tumor are distinct, the progression of a tumor inevitably involves changes in gene expression that result in differences in the complement of cell surface proteins. In addition, cancers that are classified within the same group often arise from distinct cell types that require different treatment protocols. Accordingly, the rapid identification of differences in cell surface proteins will be useful for tumor identification, staging and treatment selection, as well basic research into the mechanisms of tumor progression.

For example, diagnosis and treatment of leukemias could be substantially improved with the identification of additional cell surface markers. Acute leukemias are currently classified into those arising from lymphoid precursors (acute lymphoblastic leukemia, ALL) and those arising from myeloid precursors (acute myeloid leukemia). This classification is made primarily on the basis of lymphoid- or myeloid-specific cell surface markers, in combination with nuclear morphology, periodic acid-Schiff base staining, and detection of myeloperoxidase. Although the distinction between AML and ALL is well established, no single test is currently sufficient to establish the diagnosis. The selection of an appropriate treatment protocol depends upon the correct identification of ALL or AML. Chemotherapy for AML generally involves corticosteroids, vincristine, methotrexate, and L-asparaginase, whereas most AML regimens rely on daunorubicin and cytarabine (Pui et al. (1998) N. Engl. J. Med. 339: 605).

Several cell surface proteins are known to be useful in distinguishing ALL from AML, including CD11c, CD33, and MB-1. Recent transcriptome analysis demonstrated that an additional membrane protein, leptin receptor, is also differentially expressed (high expression in AML) (Golub et al. (1999) Science 286 (5439): 531-537). In addition, the leptin receptor may have a functional role in inhibiting apoptosis of neoplastic cells, and thus represents a target for therapeutic intervention. The identification of further distinctive membrane proteins would clearly have benefits both for diagnostics and treatment, and provide an advantage over the use of transcriptome analysis because the direct analysis of proteins takes into account any post-transcriptional regulation (Konopleva, et al. (1999) Blood 93: 1668).

In another example, a variety of secreted and cell surface proteins are used in the identification of prostate cancers. The most commonly utilized tests for prostate cancer are digital rectal examination and analysis of serum prostate specific antigen (PSA). Although PSA has been widely used as a clinical marker of prostate cancer since 1988, screening programs utilizing PSA alone or in combination with digital rectal examination have not been successful in improving the survival rate for men with prostate cancer. While PSA is specific to prostate tissue, it is produced by normal and benign as well as malignant prostatic epithelium, resulting in a high false-positive rate for prostate cancer detection. Other markers that have been used for prostate cancer detection include prostatic acid phosphatase (PAP) and prostate secreted protein (PSP). PAP is secreted by prostate cells under hormonal control. It has less specificity and sensitivity than does PSA. As a result, it is used much less now, although PAP may still have some applications for monitoring metastatic patients that have failed primary treatments. In general, PSP is a more sensitive biomarker than PAP, but is not as sensitive as PSA. Like PSA, PSP levels are frequently elevated in patients with BPH as well as those with prostate cancer. Another serum marker associated with prostate disease is prostate specific membrane antigen (PSMA). PSMA is a Type II cell membrane protein and has been identified as Folic Acid Hydrolase (FAH). Antibodies against PSMA react with both normal prostate tissue and prostate cancer tissue (Horoszewicz et al., 1987). However, PSMA may have utility in certain circumstances. PSMA is expressed in metastatic prostate tumor capillary beds (Silver et al., 1997) and is reported to be more abundant in the blood of metastatic cancer patients (Murphy et al., 1996). Recently, prostate stem cell antigen (PSCA) was identified as a cell surface protein that is overexpressed in prostate cancer cells. This marker has proven useful in diagnosing prostate cancer, and monoclonal antibodies targeting PSCA have shown some promise in treating prostate cancer in animal models.

While many cell surface and secreted molecules related to prostate cancer have been identified, clear and reliable diagnostic markers have not yet been identified. The rapid and large-scale identification of cell surface proteins from normal and cancerous prostate cancer holds great promise for the development of improved prostate cancer diagnostics and therapeutics.

Viral Infections

In general, viruses may exist in several different states within the host. The lysogenic lifecycle typically involves semi-stable incorporation of the viral genome into the host cell accompanied by an absence or relatively low level of viral reproduction. The lytic lifecycle usually involves rapid replication of the viral genome, production of viral particles, viral maturation and host cell death. Viral infection results in a change in the host cell protein production and these differences are reflected in the profile of cell surface proteins. Proteins differentially present at the cell surface may be useful as targets for antiviral therapy and may also be used in diagnosing and staging viral infections.

For example, cytomegalovirus encodes two proteins, US2 and US11 that target MHC class I and class II molecules for degradation, substantially decreasing the amount of these critical immune recognition proteins present on the membranes of infected cells (Shamu et al. (1999) J Cell Biol. 147(1): 45-58; Tomazin et al. (1999) Nat Med 5(9): 1039-43). Similarly, the Vpu protein of HIV target the host CD4 protein for destruction through a ubiquitin and proteosome-dependent pathway (Schubert et al. (1998) J. Virol. 72(3): 2280-8). Thus, many viruses alter the complement of proteins present on the surface of the host cell.

In addition, viral maturation recruits a number of viral proteins to the cell membrane for assembly into the newly forming virion. It is known that this process involves a number of host proteins, including the clathrin-mediated vesicle transport system and the ubiquitination system. We predict that a number of host proteins will re-localize to the cell surface during viral maturation. Such proteins may be functionally important in viral maturation and may therefore be suitable targets for antiviral therapy. Accordingly, the characterization of cell surface protein profiles from cells at various stages of viral infection will be a powerful method for identifying proteins useful in treatment and diagnosis of viral diseases.

Other infective states, such as intracellular bacterial pathogens and eukaryotic parasites are also anticipated to cause informative changes in cell surface protein composition.

Cell Surface Markers

Cell surface markers provided by the invention may be used in a variety of methods for the separation or characterization of cell populations. In one embodiment of the invention, sample cells can be detected and quantified using a flow cytometer. Fluorescence activated cell sorting (FACS) flow cytometry is a common technique for antibody based cell detection and separation. Typically, detection and separation by flow cytometry is performed as follows. A sample containing the cells of interest is contacted with fluorochrome-conjugated antibodies, which allows for the binding of the antibodies to one or more specific cell markers. The bound cells are washed, typically by one or more centrifugation and resuspension steps. The cells are then run through a FACS device which separates the cells based on, among other characteristics, the different fluorescence properties imparted by the cell-bound fluorochrome. FACS systems are available in varying levels of performance and ability, including multicolor analysis which is preferred in the present invention. For use of multiple cell surface markers, it is preferable to use fluorochromes with distinguishable fluorescence properties. For a general review of flow cytometry, see Parks et al., 1986, Chapter 29: Flow Cytometry and fluorescence activated cell sorting (FACS) in: Handbook of Experimental Immunology, Volume 1: Immunochemistry, Weir et al. (eds.), Blackwell Scientific Publications, Boston, Mass.

Cell surface markers may also be used in other cell detection and separation techniques. One such method is biotin-avidin based separation by affinity chromatography. Typically, such a technique is performed by incubating the sample of cells with biotin-conjugated antibodies to cell surface markers of interest, followed by contact with an avidin-coated substrate such as a column. Biotin-antibody-cell complexes bind to the column via the biotin-avidin interaction, while other cells pass through the column. The specificity of the biotin-avidin system is well suited for rapid positive detection and separation. Once isolated, the cells can be quantified and characterized as desired. Yet another method is magnetic separation using antibody-coated magnetic beads. Kemmner et al., 1992, J. Immunol. Methods 147: 197-200; Racila et al., 1998, Proc. Natl. Acad. Sci. USA 95: 4589-4594. Another exemplary cell separation methods involves the use of antibodies and protein A-coated substrates. In addition, in situ microscopy methods may be used to identify cells with the markers of interest on their surfaces.

7. Computer and Database Systems

In certain aspects the invention provides computer systems and computer-assisted methods for analyzing membrane surface proteins. A computer system of the invention may comprise a database system comprising a plurality of records reflecting membrane surface protein profiles for different samples and a user interface allowing a user to selectively view information from each profile. In preferred embodiments, the database system will comprise, in addition to membrane surface protein profiles, linked entries reflecting the nature of the sample or cells from which each profile was obtained. For example, such an entry may contain clinical information such as patient history, clinical diagnosis, clinical test results, prognosis, treatment regimen and outcome. Such an entry may also include information regarding genotype of the subject or cells from which the sample was obtained. For example, cancers typically contain a number of chromosomal abnormalities and these may be reflected in a linked database entry. With respect to viral infections, linked entries may indicate the type of viral infection. Other types of information that may be entered as linked entries include, but are not limited to, levels of various transcripts and levels of intracellular proteins.

A variety of software packages are available for data collection and analysis. Preferred data analysis systems are able to scan 2D gels and assign different colors to different fluorophores present in the gels. This permits direct comparison of differentially-labeled protein resolved on the same gel. For example, Z3 software for the analysis of 2D gels is available from Compugen Inc.

8. Membrane Surface Markers and Screening Assays for Novel Therapeutics

In yet other aspects, the invention provides methods for identifying a membrane surface protein markers. Such methods may include obtaining a cell surface protein profile from a cell type of interest, and comparing that profile to other cell types to identify distinguishing markers for the cell type of interest. For example, such methodology may be used to identify stem cell-specific cell surface markers. Such markers may then be used to enrich for cells of interest. In a further illustrative example, a marker for infection with a particular virus may be identified and used to identify subjects having infected cells. Marker proteins may be used to separate cells by Fluorescence Activated Cell Sorting (FACS) or other marker-based separation methods.

Markers and/or profiles may be used to screen for therapeutics. Cell surface proteins associated with a disease state may be diminished or eliminated by treatment with certain test compounds. Such test compounds may be useful as therapeutics for the disease state. In addition, certain test compounds may increase the presence of cell surface proteins that are normally present on healthy cells but diminished or absent in diseased cells. Such test compounds may also be useful as therapeutics. Particularly preferred therapeutics will cause the cell surface protein profile of a diseased cell to more closely resemble the cell surface protein profile of a healthy cell.

In further embodiments, the differences between healthy and unhealthy tissue samples may be analyzed to identify targets for therapeutic screening, and a screen may be designed to identify compounds that bind or otherwise affect the activity of the given target. For example, as noted above, leptin receptor is selectively overexpressed in certain leukemias. If, in fact, this overexpression leads to an increase in the level of leptin receptor present at the cell surface, therapeutics that disrupt the leptin receptor signaling pathway may be useful in treating leukemias.

In certain embodiments, a method for selecting an appropriate therapeutic for a subject is a computer-assisted method. Such a method may comprise obtaining a cell surface protein profile or measuring a marker protein in a sample from a subject. The output signal may then be compared against a database comprising output signal information from a plurality of subjects and further comprising clinical status information from a plurality of subjects. It is contemplated that one may use a computer interface to identify in the database any clinical conditions correlated with the protein profile or marker. Accordingly, one may select a targeted therapeutic to ameliorate or prevent the correlated condition.

### EXAMPLES

The invention now being generally described, it will be more readily understood by reference to the following examples, which are included merely for purposes of illustration of certain aspects and embodiments of the present invention, and are not intended to limit the invention.

### Example 1

### Tagging of Cell Surface Proteins in Live Cells with EZ-LINK NHA-SS-Biotin

One set of HeLa cells is labeled with cleavable biotin, and a second with DMSO as control.
1. Wash cells three times with cold PBS.
2. Detach cells from 4 roller bottles with 50 ml PBS/5 mM EDTA (prepared at room temperature) for 15 minutes at the incubator, while rolling. Place in a 50 ml tubes and pellet cells at 1800 rpm, 4° C. for 5 minutes. Count cells.
3. Resuspend cells from all tubes in 50 ml PBS/CM and spin down at 1800 rpm, 4° C. for 10 minutes.
4. Resuspend the cells at 25×10⁶ cells/ml in PBS/CM containing 0.5 mg/ml sulfo-biotin-NHS. Place cells in a 5 ml snap cup tube and cover with aluminum foil.
5. Incubate with gentle shaking, in the cold cabinet, for 20 minutes. Spin down cells, 1500 rpm, 4° C. for 5 minutes. Resuspend at 25×10⁶ cells/ml in 0.5 mg/ml PBS/CM containing 0.5 mg/ml sulfo-biotin-NHS. Incubate as before for 20 more minutes.
6. Transfer cell suspension to a 15 ml tube. Pellet cells as in step 5. Quench reaction by gently resuspending cells in 5 ml of 50 mM glycine in PBS/CM. Incubate with gentle shaking for 10 minutes at 4° C.
7. Wash cells three times in PBS/CM, by centrifugation as in step 5.
8. Resuspend cells in 2 ml solubilization buffer containing 50 mM Tris-HCl, pH7.6, 150 mM NaCl, 10% glycerol, 2% ASB14, 5 mM EDTA, 1 mM EGTA, 1.5 mM MgCl₂, Protease inhibitors. Cell lysis for membrane proteins is usually done with a buffer containing 0.5% Triton X-100. However, it was determined in our laboratory that ASB-14 is a better solubilizing agent.
9. Incubate on ice for 30 minutes. Spin for 20 minutes at 14,000 rpm, 4° C.
10. Transfer supernatant to a fresh tubes.
11. To each of the tubes containing the supernatant add streptavidin agarose beads (Pierce) (80 μl beads to 1 ml solubilized extract). Incubate in the thermomixer, 1400 rpm, 1 hour, 4° C.
12. Spin down beads, 1 minute, 14,000 rpm, 4° C. Aspirate off the supernatant.
13. Wash beads twice for 5 minutes, with gentle agitation, with 1 ml wash buffer 1, containing 20 mM Tris-HCl, pH7.6, 300 mM NaCl, 10% glycerol, 0.1% Triton X-100, 0.1% SDS, 1 mM EDTA, 1 mM EGTA, 1.5 mM MgCl₂, Protease inhibitors.
14. Wash once with 1 ml wash buffer 2 containing 20 mM Tris-HCl, pH7.5, 10% glycerol, 0.1% Triton X-100, 1 mM EDTA, 1 mM EGTA, 1.5 mM MgCl₂, Protease inhibitors and phosphatase inhibitors. Spin for 2 minutes at 14,000 rpm, 4° C. Discard sup.
15. To the final bead pellet add double bead volume reducing solution containing 20 mm Tris-HCl, pH7.6, 50 mM TCEP-HCL, 150 mM NaCl, protease inhibitors and incubate at room temperature for 2 hours.
16. At the end of the incubation spin at 14,000 rpm for 2 minutes. The supernatant contains the cell surface biotinylated proteins ready for analysis. The sample can be analyzed by SDS_PAGE as well as 2D electrophoresis. Since the final products contain almost exclusively integral cell surface proteins it is possible to analyze proteins by one dimension only. Furthermore, the resulting proteins can be subjected to any form of separation such as HPLC or FPLC which will be directly linked to mass spectrometry analysis.

### Example 2

### Tagging of Cell Surface Proteins in Live Cell with Alexa Fluor®488-NHS

There are several products of Alexa Fluor, each has a different emission maximum. Thus, cells can be treated differently and labeled with different emitting Alexa Fluor reagents. The detection can be done with two lasers, one that will detect one fluorophore and the other the second. An image can then be generated and the proteins that are found in both samples will give a different color then each flourphore alone.

One set of HeLa cells is labeled with Alexa Fluor®488-NHS, and a second with DMSO as control. All steps are performed on ice to prevent internalization of cell surface proteins.
1. Wash cells three times with cold PBS.
2. Detach cells from 4 roller bottles with 50 ml PBS/5 mM EDTA (prepared at room temperature) for 15 minutes at the incubator, while rolling. Place in a 50 ml tubes and pellet cells at 1800 rpm, 4° C. for 5 minutes. Count cells.
3. Resuspend cells from all tubes in 50 ml PBS/CM and spin down at 1800 rpm, 4° C. for 10 minutes.
4. Resuspend the cells at 25×10⁶ cells/ml in PBS/CM containing 0.5 mg/ml Alexa Fluor®488-NHS. Place cells in a 5 ml snap cup tube and cover with aluminum foil.
5. Incubate with gentle shaking, in the cold cabinet, for 20 minutes. Spin down cells, 1500 rpm, 4° C. for 5 minutes. Resuspend at 25×10⁶ cells/ml in 0.5 mg/ml PBS/CM containing 0.5 mg/ml Alexa Fluor®488-NHS. Incubate as before for 20 more minutes.
6. Transfer cell suspension to a 15 ml tube. Pellet cells as in step 5. Quench reaction by gently resuspending cells in 5 ml of 50 mM glycine in PBS/CM. Incubate with gentle shaking for 10 minutes at 4° C.
7. Wash cells three times in PBS/CM, by centrifugation as in step 5.
8. Aspirate supernatant gently with a Pasteur pipette hooked to the vacuum pump. Measure the volume of the cell pellet by comparing to an equivalent tube containing a known volume of water measured by a pipetman. Resuspend in 3× cell volume of ice cold lysis buffer (50 mM Tris-HCl, pH7.6, 15 mM KCl, 2 mM MgCl₂, 2 mM DTT, 1× protease inhibitor cocktail, incubate on ice for 30 minutes.
9. Subject the cells to two round of freeze-thaw cycles in liquid nitrogen-37° C. water bath to break cell membrane.
10. Remove the unbroken cells and nuclei by centrifugation, 3000×g for 10 minutes at 4° C. Remove supernatant to a clean eppendorf tube. Transfer supernatant to a fresh eppendorf tube.
11. Spin at 10,000×g for 30 minutes in an eppendorf centrifuge. Remove supernatant (cytosol), the pellet is the membrane fraction.
12. Wash the membrane to get rid of peripheral proteins. Set the thermomixer at 700 rpm and 4° C. Resuspend the membrane pellet with 45 μl lysis buffer and add 450 μl ice cold 0.1 M sodium carbonate (stored at 4° C.) containing 1× protease inhibitors. Place tubes in the thermomixer and mix for 1 hour.
13. Transfer protein mixture from step 5 to ultracentrifuge 120.1 tubes. Spin at 55,000 rpm, 4° C. for 20 minutes.
14. Remove supernatant (peripheral proteins; make sure that you remove most of the supernatant). Resuspend the membrane pellet in 400 μl ice cold 50 mM Tris-HCl, pH7.6 containing 1× protease inhibitors. Spin at 55,000 rpm, 4° C. for 20 minutes. The supernatant contains cell surface fluorescent proteins ready for analysis. The sample can be analyzed by SDSPAGE as well as 2D electrophoresis without the need for staining. The resulting proteins can be subjected to any form of separation such as HPLC or FPLC which will be directly linked to mass spectrometry analysis.

### Example 3

### Cell Surface Protein Profiling Methodologies

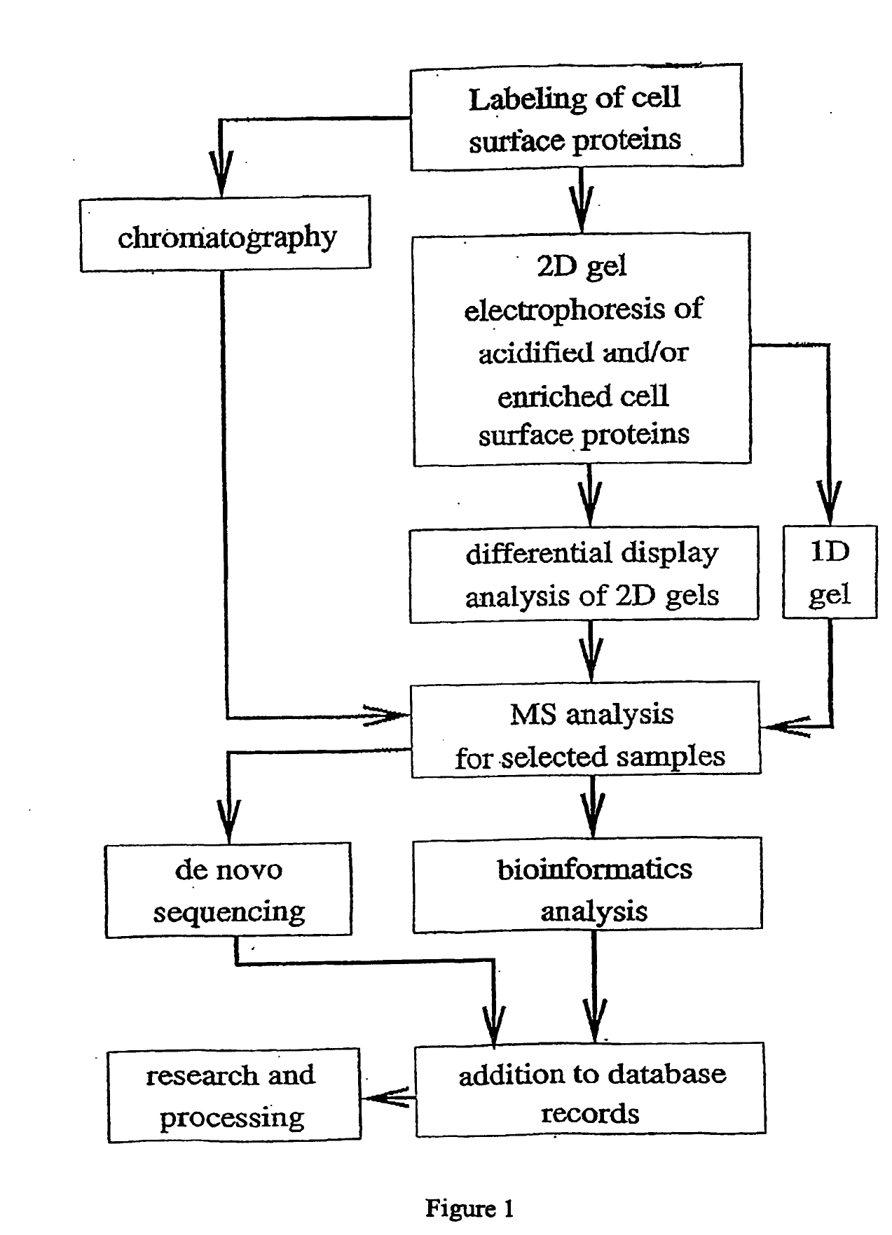

The flow chart in FIG. 1 exemplifies several possible combinations of cell surface protein labeling and identification techniques. A summary of certain aspects of the illustrated methods is set forth below.

These exemplary methods begin with a selective labeling of cell surface proteins. The labeling method, when performed using a labeling agent that binds to lysine residues, acidifies proteins, making isoelectric focusing (and thereby 2D gel electrophoresis) possible for highly basic proteins. The labeled proteins are ultimately identified by mass spectrometry analysis. Resolution of proteins for mass spectrometry may be accomplished by chromatographic separations, 2D gel electrophoresis or 1D gel electrophoresis. 2D gel electrophoresis may also be used as a part of differential display method for identifying those proteins whose expression levels change in different conditions.

MS analysis provides a wealth of information including protein sequence. This information can fed into database records and used for generating and analysing cell surface protein profiles obtained from a variety of sources.

### Example 4

### Multiple Labeling Methods for Profiling Membrane Surface Proteins

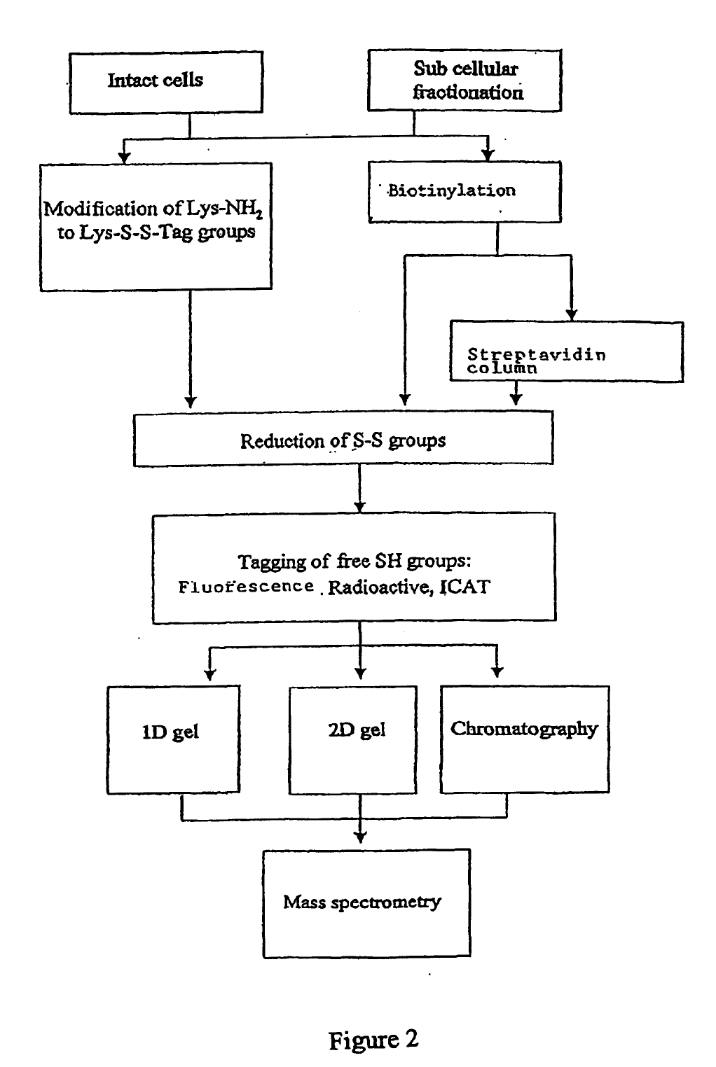

The flow chart in FIG. 2 exemplifies several possible combinations of cell surface protein labeling and identification techniques. A summary of certain aspects of the illustrated methods is set forth below.

The starting material may be either intact cells or other closed membrane structures obtained from cells, such as organelles or vesicles. Such subcellular structures may be obtained by fractionation, for example by sucrose density gradient centrifugation.

The starting material is treated with a labeling agent that reacts with amines and has a disulfide bond. In one variation, the labeling agent has a marking moiety that is biotin, which is connected to the protein binding moiety through a disulfide bond. The biotinylated cell surface proteins may be enriched by passage over a streptavidin column. Whether enriched or not, the labeled proteins can then be subjected to reducing conditions that break the disulfide bond. This process results in labeled proteins having a free thiol at positions formerly having an amine. Because amines are generally more abundant than thiols in proteins, this method makes it possible to achieve much more efficient labeling with thiol-reactive agents. This method is particularly effective with basic proteins because these proteins tend to have many amines available for modification, and the modification process neutralizes these amines rendering the proteins more tractable to analysis by isoelectric focusing. For low abundance proteins (which many membrane proteins are), thiol-reactive labeling agents often give insufficient signal because of the low number of thiols per protein. This method greatly improves the density of label and detectability of such low abundance proteins.

The modified proteins are then reacted with a second labeling agent that is reactive with thiols. The labeling agent may be fluorescent or radioactive (including ICAT reagents). These labeled proteins are then analyzed by chromatography or gel electrophoresis and ultimately identified by mass spectrometry. This data may then be fed into a data storage and analysis system.

### Example 5

### General Protocol for Membrane Surface Protein Labeling Using Amine Modifying Reagents

1. Prepare suspension of 10⁶-10⁸ cells/ml in a PBS solution (10 mM sodium phosphate, 0.15M NaCl, pH=7.4)
2. A water soluble amine modifying reagent (labeling agent) may be dissolved directly in an isotonic buffer which does not contain primary amines. Depending on solubility, the reagent may be dissolved in N′N-Dimethylformamide (DMF), anhydrous.
3. Attach amine modifying reagents to cell surface proteins, estimating a 10:1 ratio of labeling agent to membrane surface protein. This number may need to be optimized for different closed membrane surfaces. Published protocols are also available: [Prolinx: Protocol VER#5000-1; VER#5000-1; VER#1015]
   a. Incubate the cells in amine modifying reagent at 4 degrees for 1 hour.
   b. Modifier solution should be prepared fresh right before the use.
   c. Reaction conditions can vary depending on the cells and therefore may be optimized.
4. Add glycine for the removal of excess non reactive tag
5. Solubilize cell membrane using 1% triton X-100 and remove nuclear fraction.
6. Add detergent (for example SDS to 1%) for full membrane solubilization. The detergent for membrane solubilization must be compatible with the tagging reagents.
7. Separate labeled proteins using Agarose chromatography. Published protocol: [Prolinx protocol #VER1020]. Or SPM-HC separation beads. Published protocol [Prolinx protocol #Ver1026]
8. Elute the labeled proteins.
9. The labeled proteins can be concentrated from the elution solution using TCA precipitation and re-solubilization in the following buffers according to the need:
   a. Lamelly buffer for SDS page
   b. Solubilization buffer for 2D gels for example “Proteomem” from Sigma (St. Louis, Mo.)
   c. Separation in LC-MS

### Example 6

### General Protocol for Labeling Membrane Surface Glycoproteins Using a Carbohydrate Modifying Reagent

1. Prepare suspension of 10⁶-10⁸ cells/ml in a PBS solution (10 mM sodium phosphate, 0.15M NaCl, pH=7.4
2. Oxidizing the cell surface glycans. Oxidation conditions should be optimized based on the cells.
   Chemical oxidation using NaIO₄ [GlycoTrack™ Glycoprotein detection kit K-050] protocol 1a for labeling on membrane and protocol 2a for labeling in solution.
   Protocol for Labeling Cell Surfaces glycoproteins and protocol for Labeling of Glycoproteins in solution “Bioconjugate Techniques”, G T Hermanson, Academic Press 314-315 (1996).
3. Attach carbohydrate modifying reagents to cell surface glycoproteins using one of the following protocol “Bioconjugate Techniques”, G T Hermanson, Academic Press 314-315 (1996).
   a. Incubate the cells in amine modifying reagent at 4 degrees for 1 hour in the dark.
   b. Modifier solution should be prepared fresh right before the use.
   c. Reaction conditions can vary depending on the cells and therefore must be optimized.
4. Add small amount of glycerol for the removal of excess of non reactive tag
5. Solubilize cell membrane using 1% triton X-100 and remove nuclear
6. Add detergent (for example SDS to 1%) for full membrane solubilization. The detergent for membrane solubilization must be compatible with the tagging reagents.
7.
   Separate labeled proteins using Agarose chromatography. Published protocol: [Prolinx protocol #VER1020]. Or SPM-HC separation beads. Published protocol [Prolinx protocol #Ver1026]
   Alternatively reduce the disulfide bonds using DTT or other reducing agent. And then separate the proteins from the mix using size exclusion chromatography.
8.
   Elute the labeled proteins.
   Alternatively reduce the disulfide bonds using DTT or other reducing agent. And then separate the proteins from the mix using size exclusion chromatography.
   Alternatively de-glycosylate the labeled glycoprotein.
9. The proteins can be concentrated if needed from the elution solution using TCA precipitation and re-solubilization in a suitable buffer for the separation system:
   Laemmli buffer for SDS page
   Solubilization buffer for 2D gels “Proteomem” from Sigma
   Separation in LC-MS

Information and published protocols for Examples 5 and 6 may be found at the following websites and such information available as of the application filing date is herein incorporated by reference:
http://www.prolinx.com/
http://www.prozyme.com/glycopro/
http://www.hamptonresearch.com/catalog.html
http://www.europa-bioproducts.com/catagory.asp?MyVar=Kits
http://informagen.com/Resource_Informagen/Deprecated/868.html
http://www.htscreening.net/suppliers/readdet.html
http://scooter.cyto.purdue.edu/pucl_cd/flow/vol4/7_spon/biorad/tree.htm

### Example 7

### Exemplary Scheme for Synthesis of a Labeling Agent Comprising a BPA Group and a Disulfide Bond

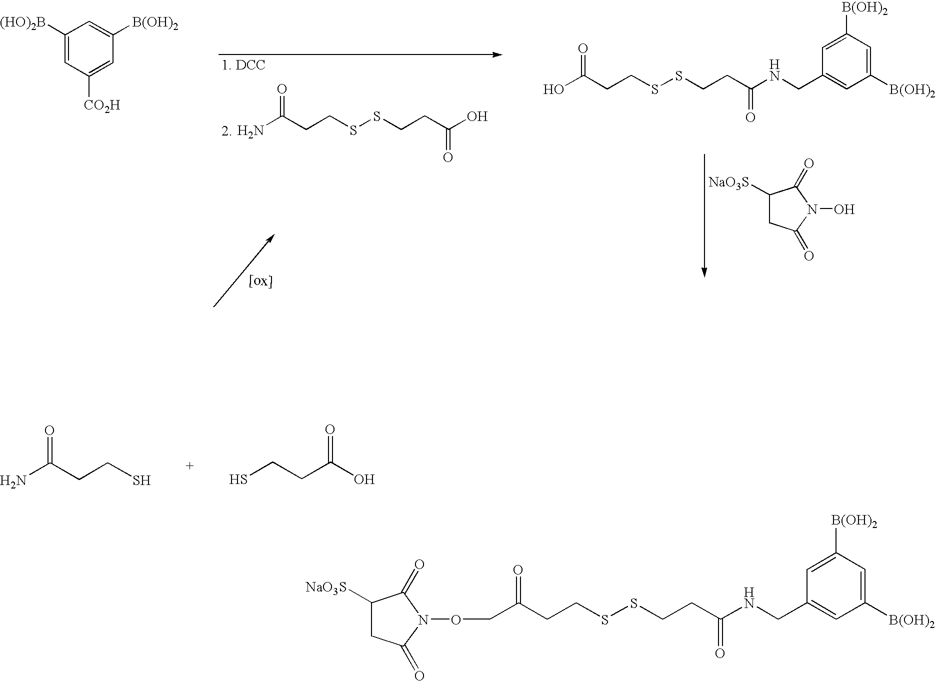

### Example 8

### Exemplary Preparation of NHS-Sulfonate Esters from Carboxylic Acids

NHS and NHS-Sulfonate are creating active esters which are used for coupling of amine to carboxile.

The attachment of NHS-S to the carboxylic acid can be done either during the coupling of the amine to the carboxyl or before the coupling.

Sulfo-NHS is reactive against amines in the same way as the NHS ester, however it's water resistant to hydrolysis is substatialy better. Since most reactions of coupling biomolecules are preformed in a water environment the advantage of using Sulfo-NHS is clear. The stability and water solubility are enabeling us to solubilize this material directly in a buffer without the need to previously solubilize in a dry organic solvent such as DMF (Dimethylformamide).

Preparation of NHS and Sulfo-NHS Active Esters for Amine Coupling: 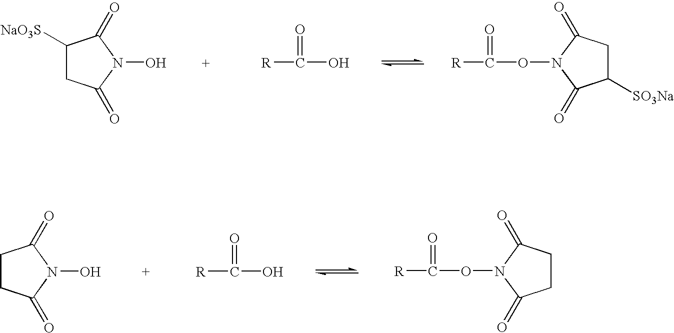
Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

1. A method of selectively labeling membrane surface proteins comprising:
(a) contacting closed membrane structures with a first labeling agent, thereby generating a plurality of primary labeled membrane surface proteins, wherein said first labeling agent comprises a disulfide bond;
(b) reducing said disulfide bond to produce primary labeled membrane surface proteins having free thiols;
(c) contacting said primary labeled membrane surface proteins with a second labeling agent, thereby generating a plurality of secondary labeled membrane surface proteins, wherein said second labeling agent comprises a thiol-reactive protein binding moiety;
(d) separating said plurality of secondary labeled membrane surface proteins from proteins not having a secondary label to obtain selectively labeled membrane surface proteins.

2. A method for generating a cell surface protein profile, comprising:
(a) contacting cells with a labeling agent, thereby generating a plurality of labeled cell surface proteins;
(b) separating said plurality of labeled cell surface proteins from unlabeled proteins; and
(c) identifying said labeled cell surface proteins separated in step (b),
wherein the cell surface protein profile comprises the identity of the labeled cell surface proteins identified in step (c) and
wherein further said labeling agent is selected from the group consisting of: 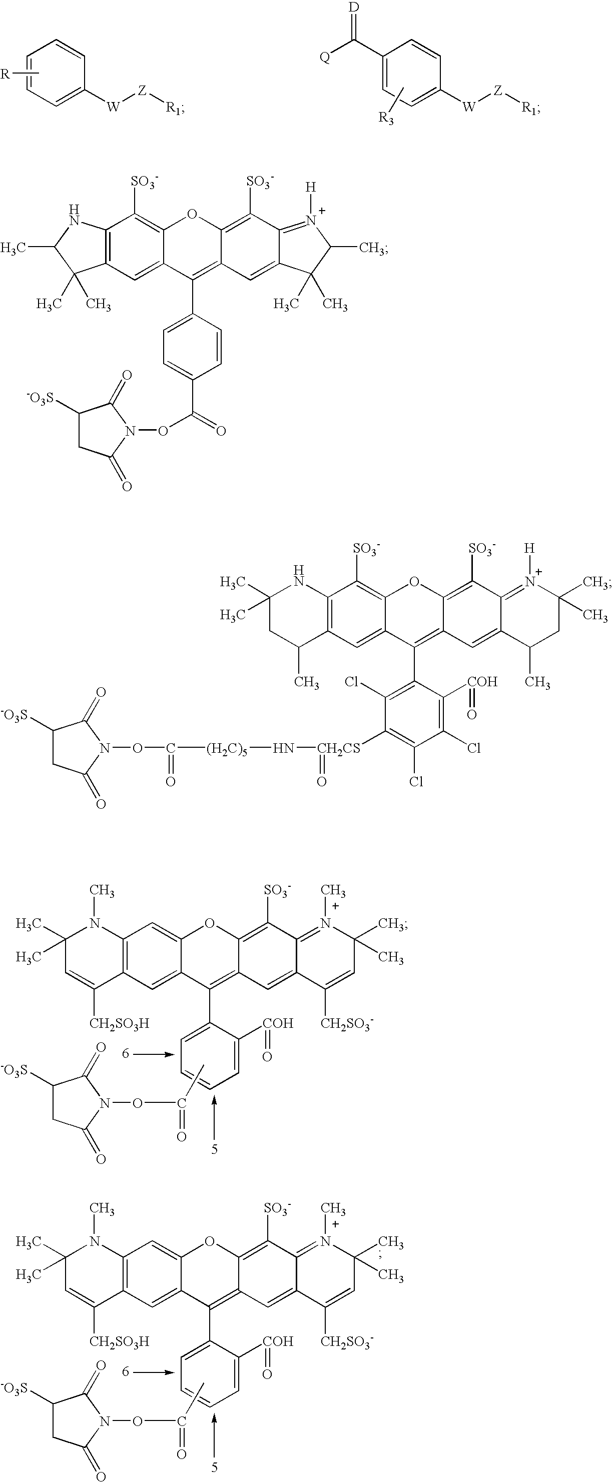 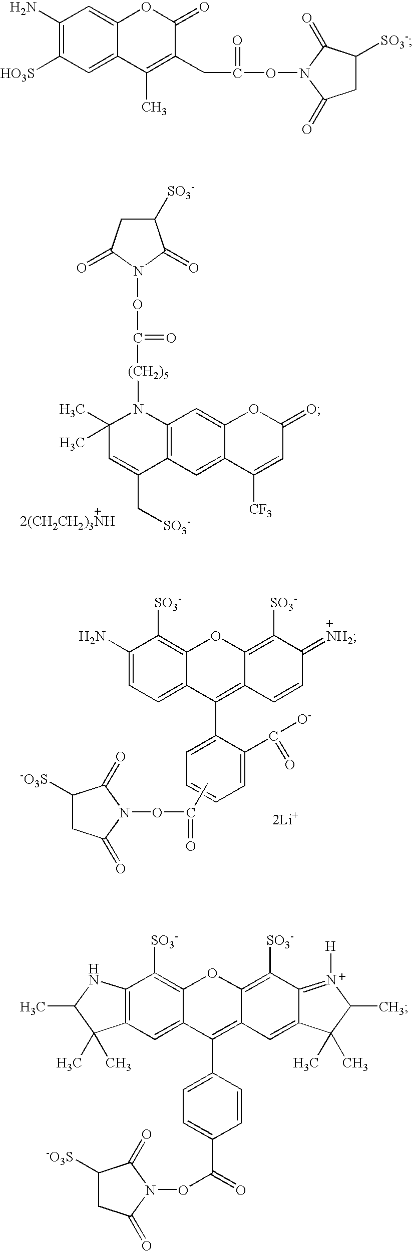 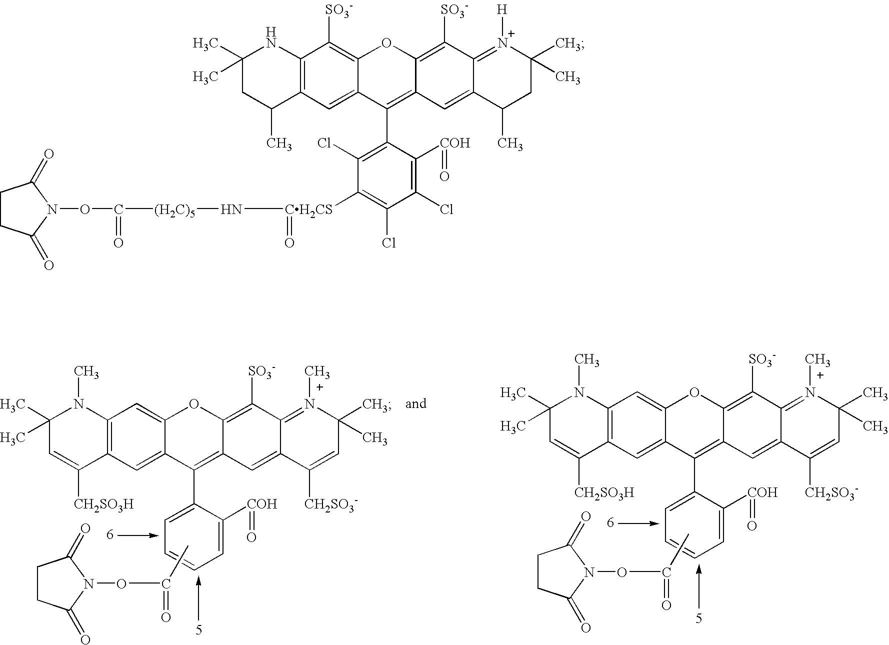
wherein
R is present 1 to 4 times and is selected from the group consisting of —B(OH)₂, 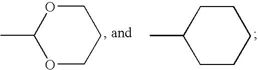
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is selected from the group consisting of N(R₂)CO, CON(R₂), N(R₂)COC(R₂)₂, CON(R₂)C(R₂)₂, O, OC(R₂)₂, S, and S(R₂)₂;
Z is selected from the group consisting of a saturated or unsaturated chain up to about 6 carbon equivalents in length, unbranched saturated or unsaturated chain of from about 6 to 18 carbon equivalents in length with at least one intermediate amide or disulfide moiety, and a polyethylene glycol chain of from about 3 to 12 carbon equivalents in length;
R₁ is a reactive electrophilic or nucleophilic moiety;
R₂ is H, alkyl, or aryl; and
R₃ is present 1 or 2 times and is OH.

3. A method for identifying cell surface proteins, comprising:
(a) contacting cells with a labeling agent, thereby generating a plurality of labeled cell surface proteins;
(b) separating said plurality of labeled cell surface proteins from unlabeled proteins; and
(c) identifying separated labeled cell surface proteins;
wherein further said labeling agent is selected from the group consisting of: 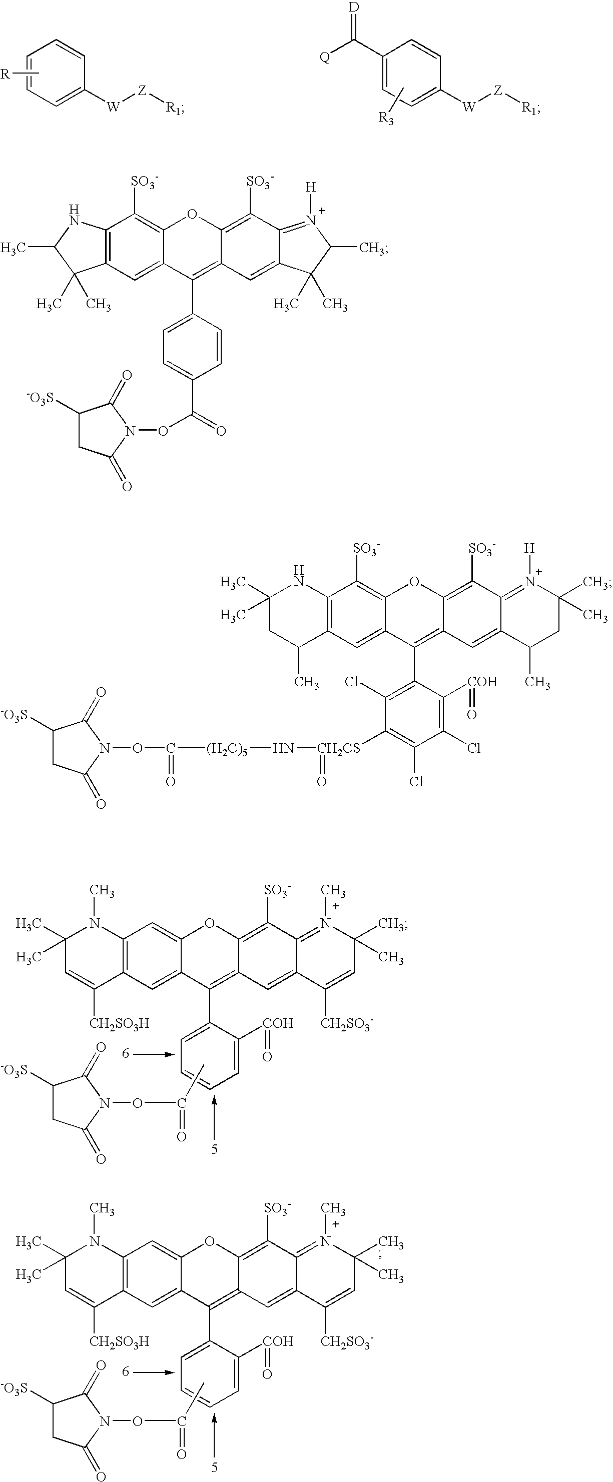 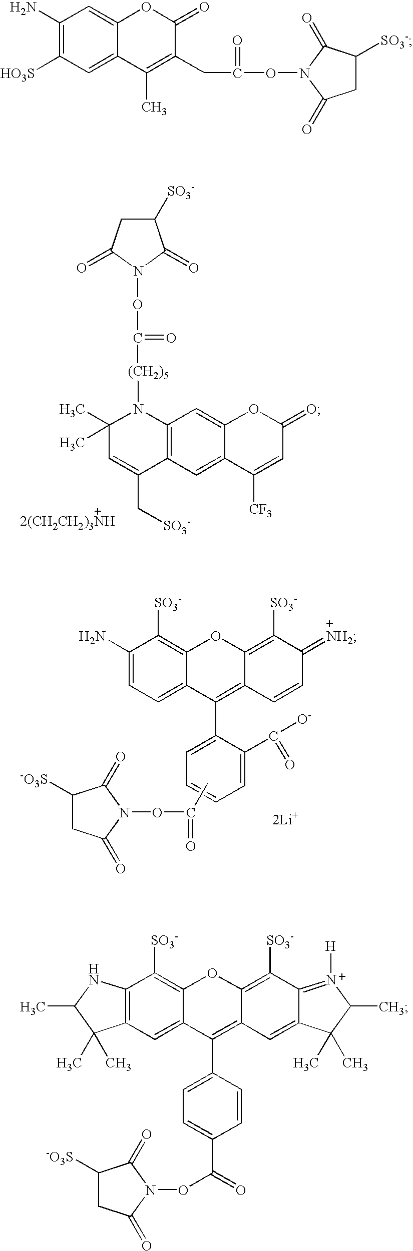 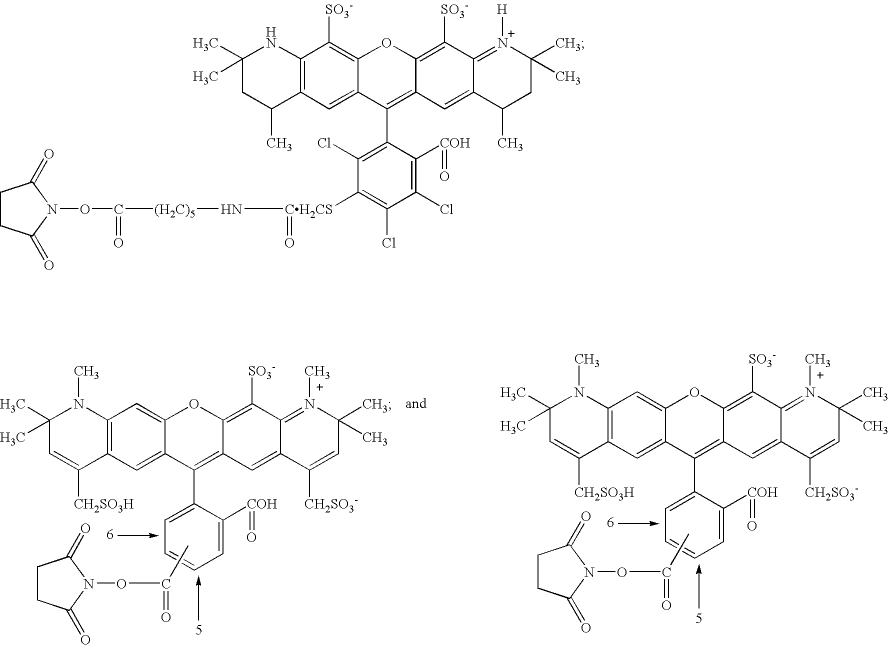
wherein
R is present 1 to 4 times and is selected from the group consisting of —B(OH)₂, 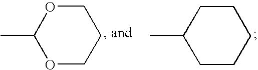
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is selected from the group consisting of N(R₂)CO, CON(R₂), N(R₂)COC(R₂)₂, CON(R₂)C(R₂)₂, O, OC(R₂)₂, S, and S(R₂)₂;
Z is selected from the group consisting of a saturated or unsaturated chain up to about 6 carbon equivalents in length, unbranched saturated or unsaturated chain of from about 6 to 18 carbon equivalents in length with at least one intermediate amide or disulfide moiety, and a polyethylene glycol chain of from about 3 to 12 carbon equivalents in length;
R₁ is a reactive electrophilic or nucleophilic moiety;
R₂ is H, alkyl, or aryl; and
R₃ is present 1 or 2 times and is OH.

4. The method of claim 1, wherein said labeling agent comprises a marking moiety and a protein binding moiety.

5. The method of claim 1, wherein said first labeling agent is selected from the group consisting of: 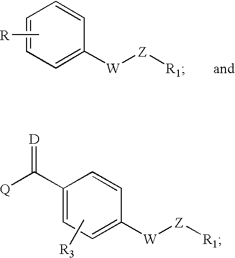
wherein
R is present 1 to 4 times and is selected from the group consisting of —B(OH)₂, 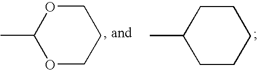
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is selected from the group consisting of N(R₂)CO, CON(R₂), N(R₂)COC(R₂)₂, CON(R₂)C(R₂)₂, O, OC(R₂)₂, S, and S(R₂)₂;
Z is an unbranched saturated or unsaturated chain of from about 6 to 18 carbon equivalents in length with at least one disulfide moiety;
R₁ is a reactive electrophilic or nucleophilic moiety;
R₂ is H, alkyl, or aryl; and
R₃ is present 1 or 2 times and is OH.

6. The method of claim 1, wherein said second labeling agent is fluorescent.

7. The method of claim 1, wherein said second labeling agent is radioactive.

8. The method of any one of claims 1, 2, or 3, wherein said cells are eukaryotic cells.

9. The method of claim 8, further comprising washing said eukaryotic cells with a divalent ion chelator to remove extracellular matrix.

10. The method of claim 9, wherein said divalent ion chelator is EDTA.

11. The method of any one of claims 1, 2, or 3, wherein said plurality of labeled cell surface proteins are separated by one-dimensional SDS polyacrylamide gel electrophoresis.

12. The method of any one of claims 1, 2, or 3, wherein said plurality of labeled cell surface proteins are separated by two-dimensional electrophoresis.

13. The method of any one of claims 1, 2, or 3, wherein said labeled cell surface proteins are identified by mass spectrometry.

14. The method of any one of claims 1, 2, or 3, wherein at least five proteins are identified.

15. A method of classifying a disease state of a test cell sample comprising:
(a) contacting cells obtained from said test cell sample with a labeling agent, thereby generating a plurality of labeled cell surface proteins;
(b) separating said plurality of labeled cell surface proteins from unlabeled proteins; and
(c) identifying said labeled cell surface proteins separated in step (b);
(d) preparing a test cell surface protein profile, said profile comprising the identity of the labeled membrane surface proteins identified in step (c);
(d) comparing said test sample cell surface protein profile to a plurality of reference cell surface protein profiles obtained from reference cell samples,
wherein said disease state of the test cell sample is classified based on similarities and differences of the test cell surface protein profile with the reference cell surface protein profiles.

16. A method of claim 15, wherein said test cell sample is suspected of having cancerous cells, and wherein at least one of said reference cell surface protein profiles is obtained from a reference cell sample having cancerous cells.

17. A method of claim 15, wherein said test cell sample is suspected of having cells infected with a virus, and wherein at least one of said reference cell surface protein profiles is obtained from a reference cell sample having cells infected with a virus.

18. A method of generating a disease-specific cell surface protein profile comprising,
(a) contacting cells obtained from a diseased cell sample with a labeling agent, thereby generating a plurality of labeled cell surface proteins;
(b) separating said plurality of labeled cell surface proteins from unlabeled proteins; and
(c) identifying said labeled cell surface proteins separated in step (b);
(d) preparing a diseased cell surface protein profile, said profile comprising the identity the labeled cell surface proteins identified in step (c);
(e) comparing said diseased cell surface protein profile to a control cell surface protein profile obtained from a control cell sample,
wherein the disease-specific cell surface protein profile comprises the identity of at least one protein that differs significantly in abundance or post-translational modification in the diseased cell sample as compared to the control cell sample.

19. A method of identifying a disorder-specific cell surface marker protein comprising,
(a) contacting cells obtained from a disordered cell sample with a labeling agent, thereby generating a plurality of labeled cell surface proteins;
(b) separating said plurality of labeled cell surface proteins from unlabeled proteins; and
(c) identifying separated labeled cell surface proteins;
(d) preparing a diseased cell surface protein profile, said profile comprising the identity of said labeled cell surface proteins identified in step (c);
(e) comparing said diseased cell surface protein profile to at least one control cell surface protein profile obtained from a control cell sample,
wherein any protein that differs significantly in abundance or post-translational modification in the diseased cell sample as compared to the control cell sample is a disease-specific cell surface marker.

20. The method of any one of claims 15, 18, or 19, wherein said labeling agent is selected from the group consisting of: 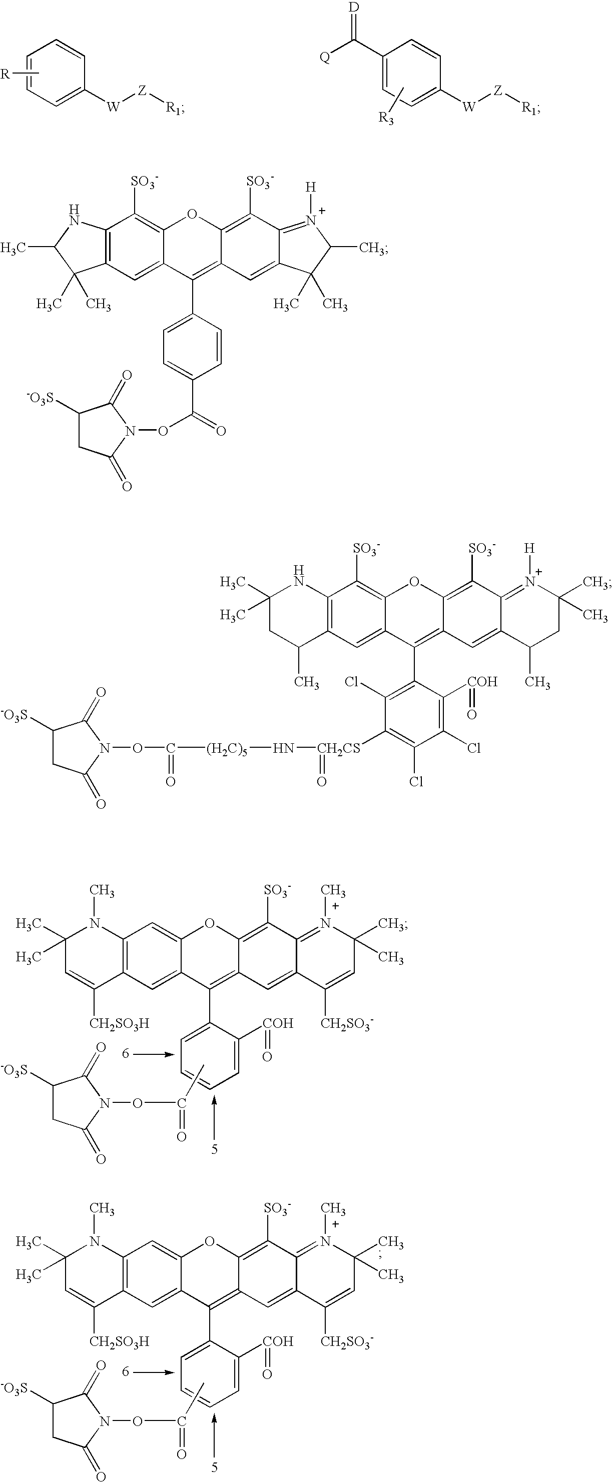 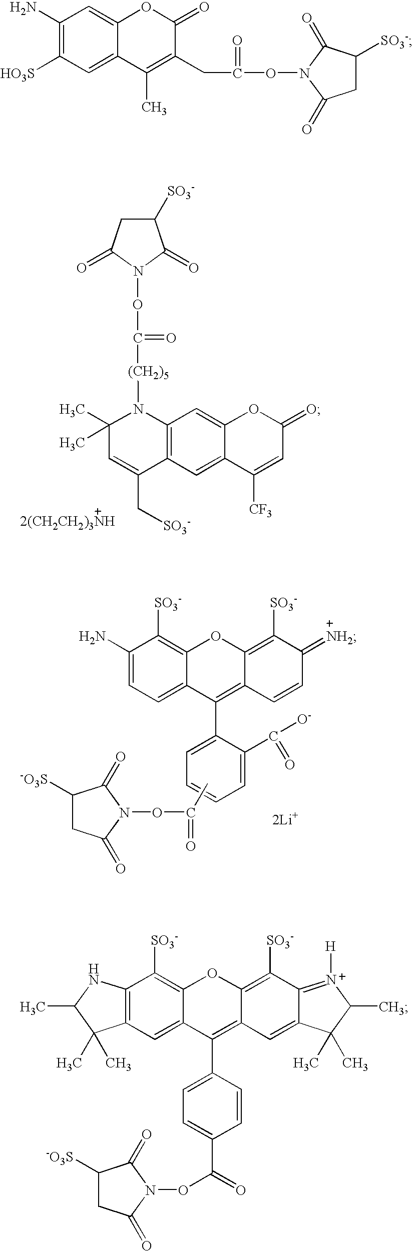 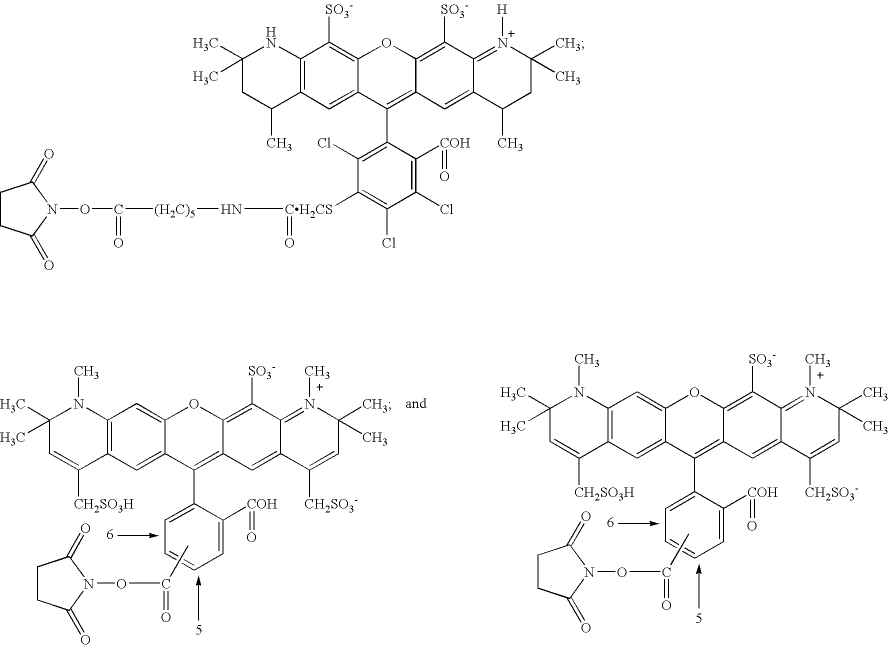
wherein
R is present 1 to 4 times and is selected from the group consisting of —B(OH)₂, 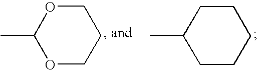
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is selected from the group consisting of N(R₂)CO, CON(R₂), N(R₂)COC(R₂)₂, CON(R₂)C(R₂)₂, O, OC(R₂)₂, S, and S(R₂)₂;
Z is selected from the group consisting of a saturated or unsaturated chain up to about 6 carbon equivalents in length, unbranched saturated or unsaturated chain of from about 6 to 18 carbon equivalents in length with at least one intermediate amide or disulfide moiety, and a polyethylene glycol chain of from about 3 to 12 carbon equivalents in length;
R₁ is a reactive electrophilic or nucleophilic moiety;
R₂ is H, alkyl, or a aryl; and
R₃ is present 1 or 2 times and is OH.

21. The method of any one of claims 15, 18, or 19, wherein said labeling agent is lectin.

22. A method of claim 15, 18 or 19, wherein said closed membrane structure is an organelle, a membrane vesicle or a cell.

23. A labeling agent represented by structure 1: 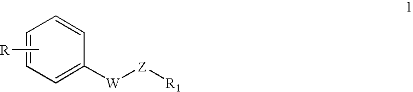
wherein:
R is present 1 to 4 times;
R is selected from the group consisting of —B(OH)₂, 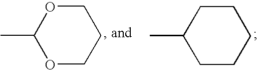
W is a linker selected from the group consisting of N(R₂)CO, CON(R₂), N(R₂)COC(R₂)₂, CON(R₂)C(R₂)₂, O, OC(R₂)₂, S, and S(R₂)₂;
Z is a spacer selected from the group consisting of an unbranched saturated or unsaturated chain of from about 6 to 18 carbon equivalents in length with at least one intermediate amide or disulfide moiety and a polyethylene glycol chain of from about 3 to 12 carbon equivalents in length;
R₁ is a reactive electrophilic or nucleophilic moiety suitable for reaction of the PDAB (phenyldiboronic acid) with a protein; and
R₂ is H, alkyl, or aryl.

24. The labeling agent of claim 23, wherein Z contains a disulfide moiety.

25. The labeling agent of claim 23, wherein R is —B(OH)₂, W is NHCO, Z is (CH₂)ₙ—S—S—(CH₂)ₙ wherein n is an integer from 1 to 6 inclusively, and R₁ is a hydrazide of structure A:

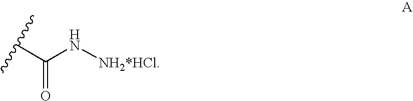

26. The labeling agent of claim 23, wherein R is 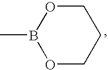
W is NHCO, Z is (CH₂)ₙ—S—S—(CH₂)ₙ wherein n is an integer from 1 to 6 inclusively, and R₁ is a hydrazide of structure A.

27. The labeling agent of claim 23, wherein R is 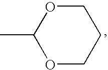
W is NHCO, Z is (CH₂)ₙ—S—S—(CH₂)ₙ wherein n is an integer from 1 to 6 inclusively, and R₁ is a hydrazide of structure A.

28. The labeling agent of claim 23, wherein R is —B(OH)₂, W is CONH, Z is (CH₂)ₙ—S—S—(CH₂)ₙ wherein n is an integer from 1 to 6 inclusively, and R₁ is a hydrazide of structure A.

29. The labeling agent of claim 23, wherein R is 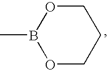
W is CONH, Z is (CH₂)ₙ—S—S—(CH₂)ₙ wherein n is an integer from 1 to 6 inclusively, and R₁ is a hydrazide of structure A.

30. The labeling agent of claim 23, wherein R is 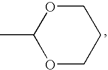
W is CONH, Z is (CH₂)ₙ—S—S—(CH₂)ₙ wherein n is an integer from 1 to 6 inclusively, and R₁ is a hydrazide of structure A.

31. The labeling agent of claim 23, wherein R is —B(OH)₂, W is CH₂NHCO, Z is (CH₂)ₙ—S—S—(CH₂)ₙ wherein n is an integer from 1 to 6 inclusively, and R₁ is a hydrazide of structure A.

32. The labeling agent of claim 23, wherein R is 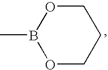
W is CH₂NHCO, Z is (CH₂)ₙ—S—S—(CH₂)ₙ wherein n is an integer from 1 to 6 inclusively, and R₁ is a hydrazide of structure A.

33. The labeling agent of claim 23, wherein R is 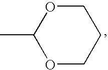
W is CH₂NHCO, Z is (CH₂)ₙ—S—S—(CH₂)ₙ wherein n is an integer from 1 to 6 inclusively, and R₁ is a hydrazide of structure A.

34. The labeling agent of claim 23, wherein R is —B(OH)₂, W is CH₂NHCO, Z is (CH₂)ₙC(O)NH(CH₂)ₙ wherein n is an integer from 1 to 6 inclusively, and R₁ is a hydroxysulfo-succinimidyl ester of structure B:

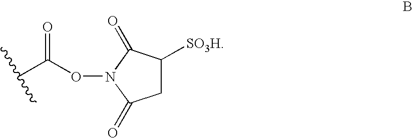

35. The labeling agent of claim 23, wherein R is —B(OH)₂, W is CH₂NHCO, Z is (CH₂)ₙ—S—S—(CH₂)ₙ wherein n is an integer from 1 to 6 inclusively, and R₁ is a hydroxysulfo-succinimidyl ester of structure B.

36. The labeling agent of claim 23, wherein R is 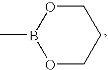
W is CH₂NHCO, Z is (CH₂)ₙC(O)NH(CH₂)ₙ wherein n is an integer from 1 to 6 inclusively, and R₁ is a hydroxysulfo-succinimidyl ester of structure B.

37. The labeling agent of claim 23, wherein R is 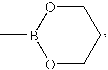
W is CH₂NHCO, Z is (CH₂)ₙ—S—S—(CH₂)ₙ wherein n is an integer from 1 to 6 inclusively, and R₁ is a hydroxysulfo-succinimidyl ester of structure B.

38. The labeling agent of claim 23, wherein R is 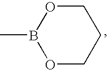
W is CONH, Z is (CH₂)₅, and R₁ is a hydroxysulfo-succinimidyl ester of structure B.

39. The labeling agent of claim 23, wherein R is —B(OH)₂, W is CONH, Z is (CH₂)₅, and R₁ is a hydroxysulfo-succinimidyl ester of structure B.

40. The labeling agent of claim 23, wherein R is 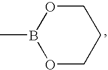
W is NHCO, Z is (CH₂)₂C(O)NH(CH₂)₅, and R₁ is a hydroxysulfo-succinimidyl ester of structure B.

41. The labeling agent of claim 23, wherein R is 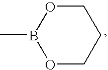
W is NHCO, Z is (CH₂)₂, and R₁ is a hydroxysulfo-succinimidyl ester of structure B.

42. A labeling agent represented by structure 2: 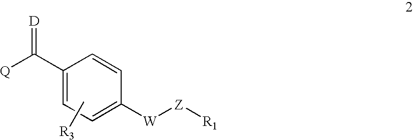
wherein:
R₃ is present 1 or 2 times and is OH;
D is selected from the group consisting of O, S, and NH;
Q is selected from the group consisting of OR₂, NHR₂, NHOR₂, and CH₂-EWG, wherein EWG is an electron withdrawing group, such as CN, COOH, etc.;
W is a linker selected from the group consisting of N(R₂)CO, CON(R₂), N(R₂)COC(R₂)₂, CON(R₂)C(R₂)₂, O, OC(R₂)₂, S, and S(R₂)₂;
Z is a spacer selected from the group consisting of unbranched saturated or unsaturated chain of from about 6 to 18 carbon equivalents in length with at least one intermediate amide or disulfide moiety and a polyethylene glycol chain of from about 3 to 12 carbon equivalents in length;
R₁ is a reactive electrophilic or nucleophilic moiety; and
R₂ is H, alkyl or aryl.

43. The labeling agent of claim 42, wherein Z contains a disulfide moiety.

44. The labeling agent of claim 42, wherein R is present one time W is NHCO, Z is (CH₂)ₙ—S—S—(CH₂)ₙ wherein n is an integer from 1 to 6 inclusively, Q is OR₂ and R₁ is a hydrazide of structure A:

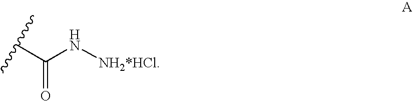

45. The labeling agent of claim 42, wherein R is present one time, W is NHCO, Z is (CH₂)ₙ—S—S—(CH₂)ₙ wherein n is an integer from 1 to 6 inclusively, Q is NHOR₂, and R₁ is a hydrazide of structure A.

46. The labeling agent of claim 42, wherein R is present two times, W is NHCO, Z is (CH₂)ₙ—S—S—(CH₂)ₙ wherein n is an integer from 1 to 6 inclusively, Q is OR₂, and R₁ is a hydrazide of structure A.

47. The labeling agent of claim 42, wherein R is present two times, W is NHCO, Z is (CH₂)ₙ—S—S—(CH₂)ₙ wherein n is an integer from 1 to 6 inclusively, Q is NHOR₂, and R₁ is a hydrazide of structure A.

48. The labeling agent of claim 42, wherein R is present one time, W is CONH, Z is (CH₂)ₙ—S—S—(CH₂)ₙ wherein n is an integer from 1 to 6 inclusively, Q is OR₂, and R₁ is a hydrazide of structure A.

49. The labeling agent of claim 42, wherein R is present one time, W is CONH, Z is (CH₂)ₙ—S—S—(CH₂)ₙ wherein n is an integer from 1 to 6 inclusively, Q is NHOR₂, and R₁ is a hydrazide of structure A.

50. The labeling agent of claim 42, wherein R is present two times, W is CONH, Z is (CH₂)ₙ—S—S—(CH₂)ₙ wherein n is an integer from 1 to 6 inclusively, Q is OR₂, and R₁ is a hydrazide of structure A.

51. The labeling agent of claim 42, wherein R is present two times, W is CONH, Z is (CH₂)ₙ—S—S—(CH₂)ₙ wherein n is an integer from 1 to 6 inclusively, Q is NHOR₂, and R₁ is a hydrazide of structure A.

52. The labeling agent of claim 42, wherein R is present one time W is NHCO, Z is (CH₂)ₙ—S—S—(CH₂)ₙ wherein n is an integer from 1 to 6 inclusively, Q is OR₂ and R₁ is a hydrazide of structure B:

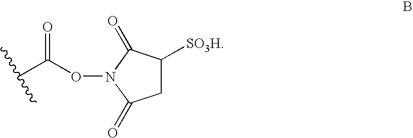

53. The labeling agent of claim 42, wherein R is present one time, W is NHCO, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

54. The labeling agent of claim 42, wherein R is present two times, W is NHCO, Z is (CH₂)ₙ—S—S—(CH₂)ₙ wherein n is an integer from 1 to 6 inclusively, Q is OR₂, and R₁ is a hydroxysulfo-succinimidyl ester of structure B.

55. The labeling agent of claim 42, wherein R is present two times, W is NHCO, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

56. The labeling agent of claim 42, wherein R is present one time, W is CONH, Z is (CH₂)ₙ—S—S—(CH₂)ₙ wherein n is an integer from 1 to 6 inclusively, Q is OR₂, and R₁ is a hydroxysulfo-succinimidyl ester of structure B.

57. The labeling agent of claim 42, wherein R is present one time, W is CONH, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

58. The labeling agent of claim 42, wherein R is present two times, W is CONH, Z is (CH₂)ₙ—S—S—(CH₂)ₙ wherein n is an integer from 1 to 6 inclusively, Q is OR₂, and R₁ is a hydroxysulfo-succinimidyl ester of structure B.

59. The labeling agent of claim 42, wherein R is present two times, W is CONH, Z is (CH₂)ₙ—S—S—(CH₂)ₙ wherein n is an integer from 1 to 6 inclusively, Q is NHOR₂, and R₁ is a hydroxysulfo-succinimidyl ester of structure B.

60. The method of claim 2, further comprising:
affixing to a solid substrate an agent that binds to the marking moiety of the labeling reagent to generate a affinity-prepared substrate; and contacting the affinity-prepared substrate with the labeled membrane surface proteins, thereby generating an array of membrane surface proteins affixed to a solid substrate.

61. The method of claim 60, further comprising:
performing a mass spectrometry analysis of a plurality of the membrane surface proteins affixed to the solid surface.

62. A linking agent represented by the structure:

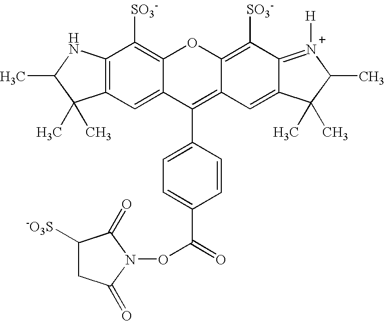

63. A linking agent represented by the structure:

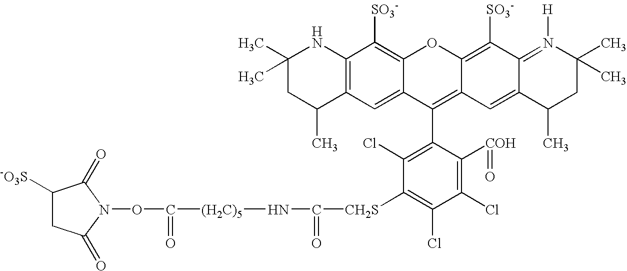

64. A linking agent represented by the structure:

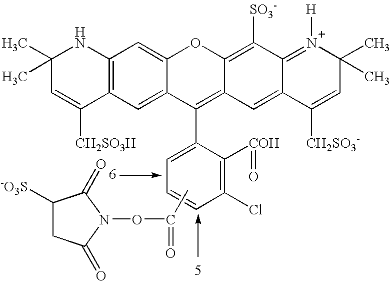

65. A linking agent represented by the structure:

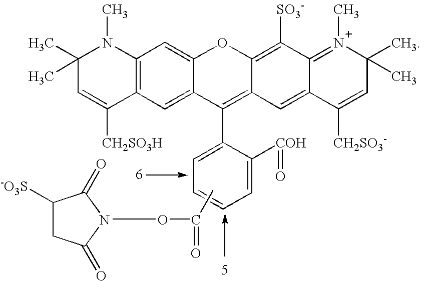

66. A linking agent represented by the structure:

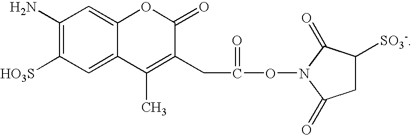

67. A linking agent represented by the structure:

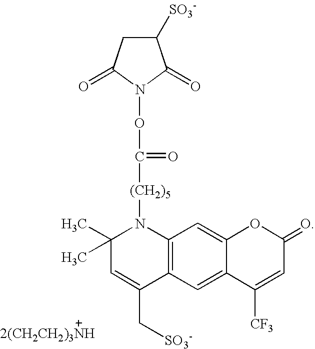

68. A linking agent represented by the structure: [chemical structure]
